(19) United States
(12) Patent Application Publication
(10) Pub. No.: US 2019/0114390 A1
(43) Pub. Date: Apr. 18, 2019

(54) DRUG REPURPOSING BASED ON DEEP EMBEDDINGS OF GENE EXPRESSION PROFILES

(71) Applicant: BioAge Labs, Inc., Richmond, CA (US)

(72) Inventors: Yonatan Nissan Donner, Sunnyvale, CA (US); Kristen Patricia Fortney, Crockett, CA (US); Stéphane Mathieu Victor Kazmierczak, Saint-Germain (FR)

(21) Appl. No.: 16/160,457

(22) Filed: Oct. 15, 2018

Related U.S. Application Data
(60) Provisional application No. 62/571,981, filed on Oct. 13, 2017.
(60) Provisional application No. 62/644,294, filed on Mar. 16, 2018.

(51) Int. Cl. G06F 19/18; G06F 19/20; G06F 19/24; G06F 19/26; G06F 19/28; G06F 17/18; C12Q 1/6869

(52) U.S. Cl. CPC G06F 19/18; G06F 19/20; G06F 19/24; C12Q 2600/106; G06F 19/28; G06F 17/18; C12Q 1/6869; G06F 19/26

(57) ABSTRACT

A deep learning model measures functional similarities between compounds based on gene expression data for each compound. The model receives an unlabeled expression profile for a query perturbagen including transcription counts of a plurality of genes in a cell affected the query perturbagen. The model extracts an embedding of the expression profile. Using the embedding of the query perturbagen and embeddings of known perturbagens, the model determines a set of similarity scores, each indicating a likelihood that a known perturbagen has a similar effect on gene expression as the query perturbagen. The likelihood, additionally, provides a prediction that the known perturbagen and query perturbagen share pharmacological similarities. The similarity scores are ranked and, from the ranked set, at least one candidate perturbagen is determined to be pharmacologically similar to the query perturbagen. The model may further be applied to determine similarities in structure and biological protein targets between perturbagens.

### CROSS REFERENCE TO RELATED APPLICATIONS

This application claims the benefit of U.S. Provisional Application No. 62/571,981, filed on Oct. 13, 2017, and U.S. Provisional Application No. 62/644,294, filed on Mar. 16, 2018, both of which are incorporated herein by reference in their entirety for all purposes.

### BACKGROUND

### Field of Art

The disclosure relates generally to a method for drug repurposing, and more specifically, drug repurposing based on gene expression data.

### Description of the Related Art

Conventional drug repurposing methods rely on the notion that two compounds are more likely to have pharmacological similarity if the two are structurally similar (e.g., if they share chemical substructures) which is easily measurable for any pair of compounds. However, when used alone measures of structural similarity provide limited insight. In particular, compounds that share pharmacological similarities may be chemically diverse and small changes in structure may have dramatic effects on gene expression and function within a cell. More useful notions of compound similarity, for example side-effect similarity and target similarity, are only available for a small subset of compounds.

One approach, which provides insight into pharmacological similarities independent of structural similarities, is to consider compounds be similar based on their impact on cellular gene expression. However, standard measures for this approach only poorly predict pharmacological similarities between compounds. Accordingly, there exists a need for an effective way to predict pharmacological similarities between compounds based on gene expression data.

### SUMMARY

Described is a method of drug repurposing which implements a deep learning model to determine pharmacological similarities between compounds, one class of “perturbagens,” based on gene expression data of cells affected by the perturbagens. The model applies deep learning techniques to develop a metric of compound functional similarity. In one embodiment, this may be used to inform in silico drug repurposing. The model includes a densely connected architecture which can be trained without convolutions. The model is trained using a large training dataset of the effect of perturbagens on cellular expression profiles labeled with a known perturbagen and the functional properties associated with the known perturbagen. After training, the model is evaluated using a hold-out dataset of further labeled expression profiles.

The model receives an expression profile of a cell affected by a query perturbagen with unknown pharmacological properties and generates an embedding of the expression profile. Similarity scores are determined between the extracted embedding of the query perturbagen and embeddings for each of a set of known perturbagens. A similarity score between a query perturbagen and a known perturbagen indicates a likelihood that a known perturbagen has a similar effect on gene expression in a cell as the query perturbagen. The similarity scores may be ranked based on the indicated likelihood of similarity, and from the ranked set at least one of the known perturbagen is determined to be a candidate perturbagen matching the query perturbagen. The pharmacological properties associated with one or more candidate perturbagens may be assigned to the query perturbagen, confirming the pharmacologic similarity determined by the model.

In one embodiment, an expression profile is received for a query perturbagen including transcription counts of a plurality of genes in a cell affected by the query perturbagen. The expression profile is input to a trained model to extract an embedding comprising an array of features comprising corresponding feature values. The embedding of the query perturbagen is used to calculate similarity scores between the query perturbagen and the known perturbagens. Each similarity score indicates a likelihood that a known perturbagen has a similar effect on gene expression in a cell as the query perturbagen. The similarity scores are ranked based on their magnitudes, upon the basis of which at least one candidate perturbagen is determined to match the query perturbagen.

In another embodiment, a set of known perturbagens are accessed, wherein each known perturbagen is associated with at least one functional property describing an effect on gene expression in a cell. From the set of known perturbagens, a first perturbagen is selected to be a query perturbagen. For the query perturbagen, an embedding is accessed comprising feature values. Using the embedding of the query perturbagen and embeddings of known perturbagens, similarity scores are computed indicating likelihoods that each known perturbagen of the accessed set has a similar effect on gene expression in a cell as the query perturbagen. Based on the similarity scores, at least one candidate perturbagen is determined to match the query perturbagen and the functional properties associated with the candidate perturbagens are used to supplement the functional properties associated with the query perturbagen.

The described approach accurately and effectively predicts pharmacological similarities between perturbagens without requiring insight into structural similarities. Evaluation of the model's performance indicated that the predictions of the model based on gene expression data may be implemented as a supplement to existing metrics of structural similarity to provide more accurate insights into the structure-function relationships of various compounds.

### BRIEF DESCRIPTION OF DRAWINGS

FIG. 1 is a high-level block diagram of a system environment, according to one or more embodiments.

FIG. 2 shows an overview of applications of an embedding extracted from an expression profile of a cell, according to one or more embodiments.

FIG. 3 is a high-level block diagram of the profile analysis module, according to one or more embodiments.

FIG. 4 shows a flow chart of the process for determining functional properties associated with a query perturbagen, according to one or more embodiments.

FIG. 5 shows an exemplary neural network maintained by the model, according to one or more embodiments.

FIG. 6A shows an exemplary expression profile of a cell affected by a perturbagen, according to one or more embodiments.

FIG. 6B shows an exemplary embedding, according to one or more embodiments.

FIG. 7 shows an exemplary set of similarity scores computed between an embedding of a query perturbagen and embeddings of a set of known perturbagens, according to one or more embodiments.

FIG. 8 shows an exemplary training data set of known perturbagens, according to one or more embodiments.

FIG. 9 shows a flow chart of the process for internally evaluating the performance of the model, according to one or more embodiments.

FIGS. 10-14 are diagrams characterizing and analyzing the data used for evaluating the embedding extracted by the model, according to one or more embodiments.

The figures depict various embodiments of the presented invention for purposes of illustration only. One skilled in the art will readily recognize from the following discussion that alternative embodiments of the structures and methods illustrated herein may be employed without departing from the principles described herein.

### DETAILED DESCRIPTION

### I. Overview

A model is implemented to develop a metric of compound functional similarity to inform in silico drug repurposing. The model determines an embedding from an L1000 expression profile of a cell affected by a perturbagen into a multidimensional vector space. As described herein, an embedding refers to mapping from an inputs space (i.e., a gene expression profile) into a space in which another structure exists (i.e., the metric of functional similarity). A perturbagen refers to a compound or genetic or cellular manipulation which, when introduced to a cell, affects gene transcription within the cell, for example by upregulating or downregulating the production of RNA transcripts for a given gene. Accordingly, the model receives an expression profile (sometimes referred to simply as a “profile”) of a cell affected by a perturbagen for which pharmacological properties are not known, or a “query perturbagen.” The system accesses expression profiles of cells affected by perturbagens for which pharmacological properties are known, or “known perturbagens.”

The embeddings extracted from the query perturbagen and the known perturbagens are used to calculate similarity scores between the query perturbagen and each of the known perturbagens. Each of the determined similarity scores describe a likelihood that a query perturbagen shares functional properties, at least in part, with one of the known perturbagens. Accordingly, perturbagens are determined to be similar if they impact cellular gene expression in the same way. The query perturbagen is assigned functional properties associated with one or more of the most similar known perturbagens and, in some embodiments, is further supplemented with structural and pharmacological properties based on the assigned functional properties. As will be described herein, structural properties describe a relationship between the functional properties and the molecular structure of the perturbagen. Additionally, pharmacological properties describe specific biological targets affected by the perturbagen and how they are affected by the perturbagen.

### II. Computing Environment

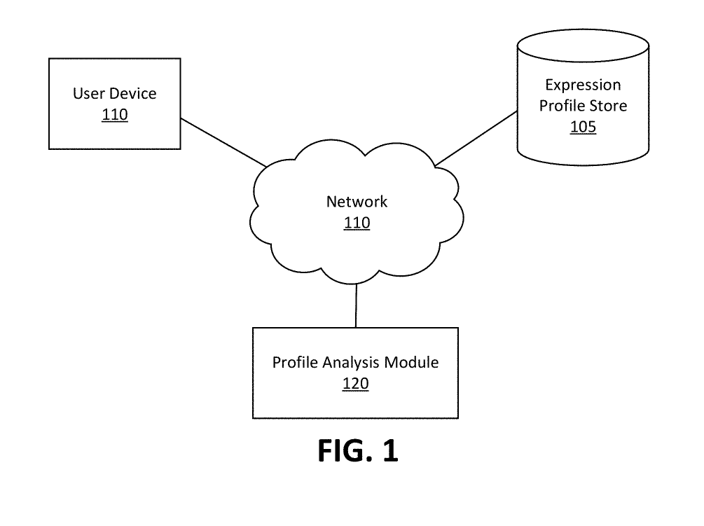

FIG. 1 is a high-level block diagram of a system environment for a compound analysis system, according to one or more embodiments. The system environment illustrated in FIG. 1 comprises an expression profile store 105, a network 110, a profile analysis module 120, and a user device 130. In alternate configurations, different and/or additional components may be included in the system environment. Additionally, the system environment may include any number of expression profile stores 105 and user devices 130.

An expression profile store 105 stores expression profiles for known and query perturbagens to be used as inputs to a deep learning model. The model is trained on a large dataset of expression profiles for cellular perturbations, with no labels other than the identities of the perturbagens applied to each sample. Because large datasets of gene expression profiles of chemically or genetically perturbed cells are now available, the model is able to implement deep learning techniques to address the problem of functional similarity between perturbagens. In one embodiment, the expression profile store 105 stores gene expression data from drug-treated cells of the Library of Integrated Network-based Cellular Signatures (LINCS) dataset, which comprises more than 1.5M expression profiles from over 40,000 perturbagens. LINCS data were gathered using the L1000 platform which measures the expression of 978 genes, each expression stored within the expression profile store 105 comprises gene expression data for the same 978 landmark genes. Despite only describing the expression of 978 genes, the LINCS dataset captures a majority of the variance of whole-genome profiles at reduced cost relative to whole genome or whole exome sequencing.

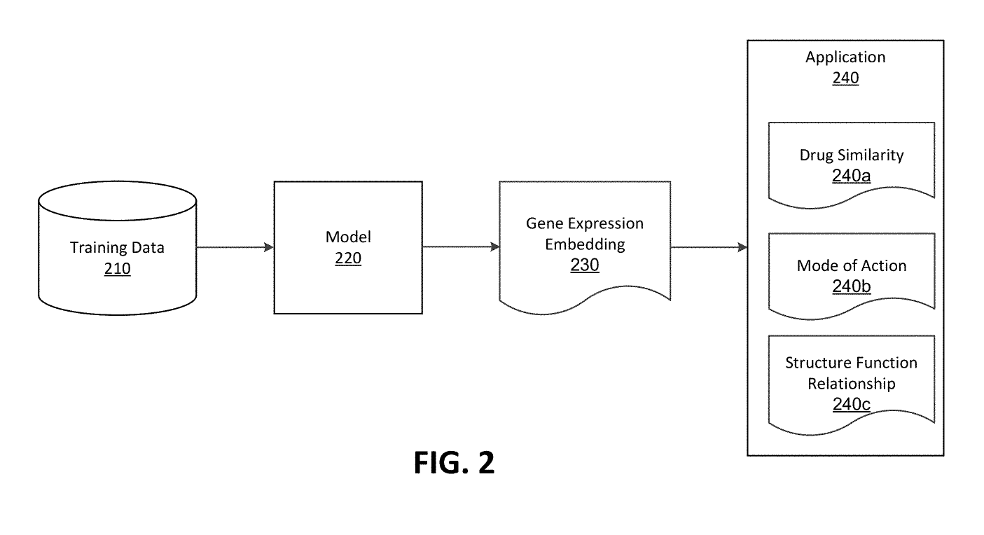

Expression profiles for both unlabeled query perturbagens and for labeled known perturbagens are accessed from the expression profile store by the profile analysis module 120. The profile analysis module 120 trains a deep learning model to extract embeddings from expression profiles labeled with known perturbagens. Once trained, the profile analysis module 120 uses the model to extract embeddings from expression profiles of unlabeled query perturbagens. The profile analysis module 120 uses extracted embeddings of the query and known perturbagens to determine a similarity score between the query perturbagen and each of the known perturbagens accessed from the expression profile store 105. Based on the determined similarity scores, the profile analysis module 120 determines a subset of the known perturbagens most similar to the query perturbagen, hereafter referred to as “candidate perturbagens.” From the known therapeutic, pharmacological, and structural properties of the candidate perturbagens, the profile analysis module 120 determines which of those properties, if not all, would be accurately associated with the query perturbagen and assigns them to the query perturbagen. The profile analysis module 120 is further described with reference to FIG. 2.

The user device 130 is a computing device capable of receiving user input as well as transmitting and/or receiving data via the network 110. In one embodiment, a user device 130 is a conventional computer system, such as a desktop or a laptop computer, which executes an application (e.g., a web browser) allowing a user to interact with data received from the profile analysis module 120. Alternatively, a user device 130 is a smart phone, a personal digital assistant (PDA), a mobile telephone, a smartphone, or another suitable device. In another embodiment, a user device 130 interacts with the profile analysis module 120 through an application programming interface (API) running on a native operating system of the user device 130, such as IOS® or ANDROID™.

Interactions between the content provider system 130, the user device 120, and the online system 140 are typically performed via the network 110, which enables communication between the user device 120, the content provider system 130, and the online system 140. In one embodiment, the network 110 uses standard communication technologies and/or protocols including, but not limited to, links using technologies such as Ethernet, 802.11, worldwide interoperability for microwave access (WiMAX), 3G, 4G, LTE, digital subscriber line (DSL), asynchronous transfer mode (ATM), InfiniBand, and PCI Express Advanced Switching. The network 110 may also utilize dedicated, custom, or private communication links. The network 110 may comprise any combination of local area and/or wide area networks, using both wired and wireless communication systems.

### III. Basic Use Case and Computer Logic Components

FIG. 2 shows a high-level overview of applications of an embedding extracted from an expression profile of a cell, according to one or more embodiments. A training data 210 comprising a set of expression profiles labeled with a known perturbagen is used to iteratively train a model 220. During training, the model 220 extrapolates latent correlations between the expression profiles of cells and pharmacological effects of a perturbagen on the cell.

Once trained, the model 220 receives an expression profile for a query perturbagen, for example gene expression data of a cell affected by an unknown perturbagen, and generates a gene expression embedding 230 comprising a number of feature values representing gene expression data in the reduced multidimensional vector space. The gene expression embedding 230 of the query perturbagen and the embedding of each of a set of known perturbagens are used to determine a set of similarity scores. Each similarity score describes a measure of similarity in pharmacological effect between the query perturbagen one of the known perturbagens. The generated embedding 230 may be analyzed through several different applications 240 to characterize the query perturbagen.

In one embodiment, based on the likelihood that the query perturbagen is similar to a candidate perturbagen, the query perturbagen may be analyzed for insight into drug similarities 240a. The functional properties of candidate perturbagens within a threshold level of similarity to the query perturbagen may be propagated and assigned to the query perturbagen. Additionally, based on pharmacological similar candidate perturbagens, the profile analysis module 120 may determine insight into the mode of action 240b of the query perturbagen based on the pharmacological effects of a candidate perturbagen. The structure-function relationship 240c may be characterized based on a combination of the similarity scores determined from the embeddings and an existing metric for structural similarity between the two perturbagens, for example Tanimoto coefficients for extended connectivity fingerprints.

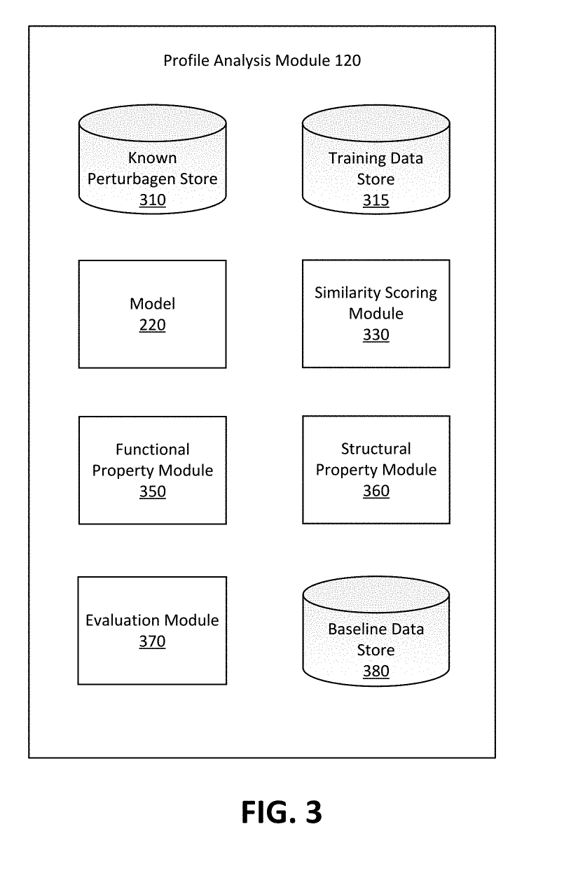

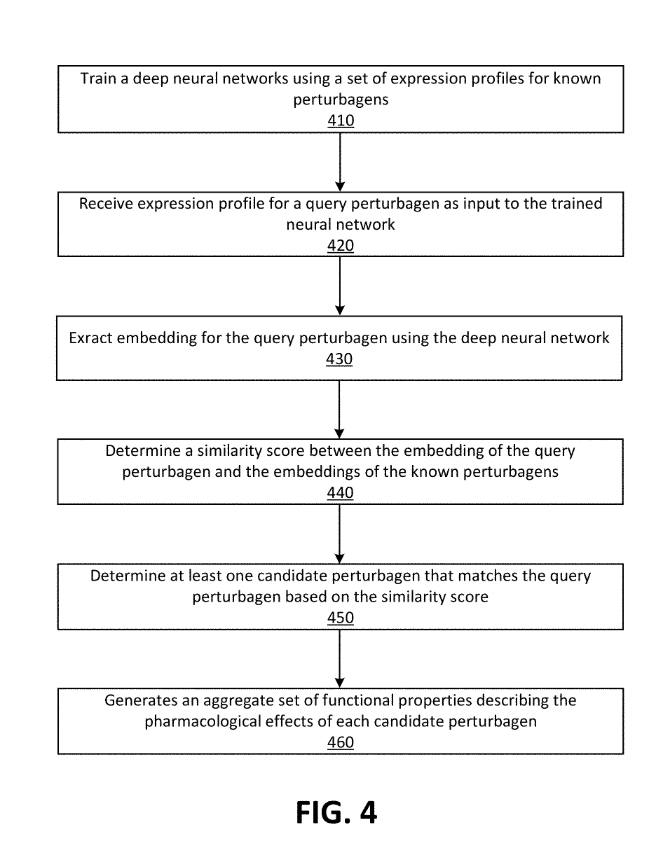

FIG. 3 is a block diagram of the system architecture for the profile analysis module 120 according to an embodiment. FIG. 4 shows a flow chart of an example process carried out by the profile analysis module 120, according to one embodiment. These two figures will be discussed in parallel for brevity. The profile analysis module 120 includes a known perturbagen store 310, a training data store 315, a model 220, a similarity scoring module 330, a functional property module 350, a structural property module 360, an evaluation module 370, and a baseline data store 380. In alternate configurations, different and/or additional components may be included in the profile analysis module 120.

The known perturbagen store 310 contains a subset of the expression profiles accessed from the expression profile store 105. In one embodiment, the accesses expression profiles are separated into a set of labeled expression profiles and unlabeled expression profiles. The labeled expression profiles which describe effects on gene expression caused by a known perturbagen are stored in the known perturbagen store 310. The remaining unlabeled expression profiles, which represent query perturbagens, are input to the trained model.

Expression profiles stored within the known perturbagen store 310 are further categorized into a training dataset, stored within training data store 315. For example, the training dataset may be comprised of 80% of the profiles within the known perturbagen store 310 and the hold-out dataset comprised of the remaining 20% of the profiles. The training dataset is used to train 410 the model 220, whereas the hold-out dataset is used to evaluate the model 220 after being trained. In one implementation, the training dataset comprises data received from the LINCS dataset and the model is trained using Phase I and II level 4 data for a total of 1,674,074 samples corresponding to 44,784 known perturbagens. After removing 106499 control samples, 1,567,575 samples corresponding to 44,693 perturbagens remained. As described above, each sample includes gene expression data for the 978 landmark genes without imputations.

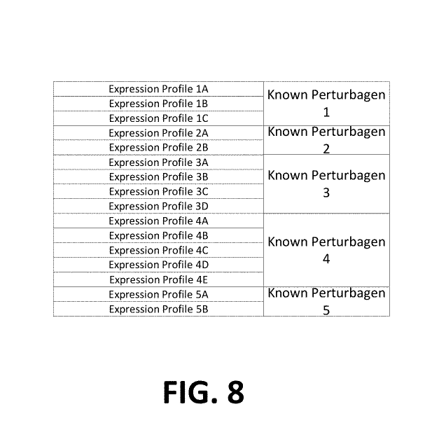

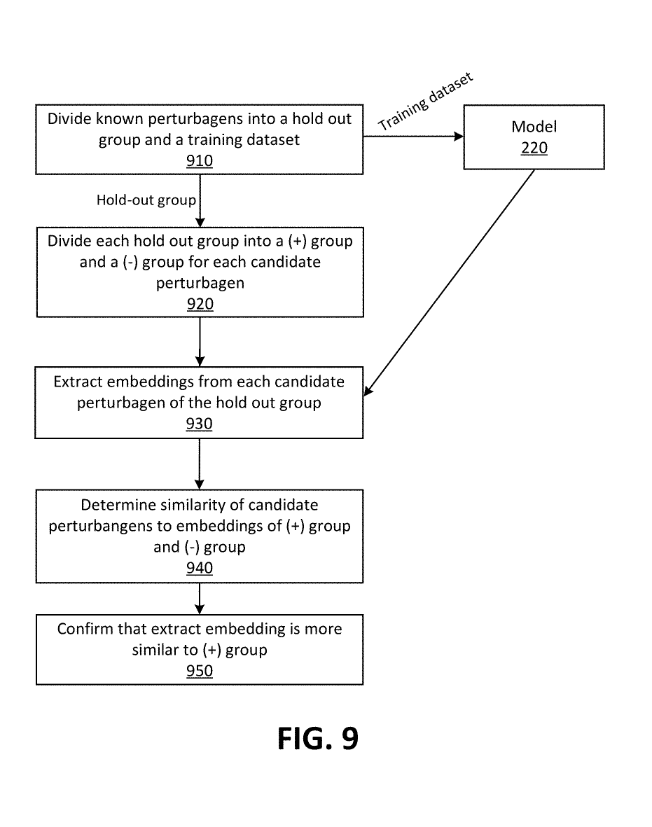

The training 410 of the model 220 using the training dataset will be further described with reference to FIG. 8 and the evaluation of the model 220 using the hold-out dataset will be further described with reference to FIG. 9. By separating the hold-out dataset from the training dataset, the profile analysis system 120 effectively evaluates the accuracy of the model by not testing the model on the same data used to train the model.

Once trained, the model 220 receives 420 an expression profile for a query perturbagen and extracts 430 an embedding from the query perturbagen. To extract an embedding, the model 220 implements a deep neural network to generate the embeddings, which may be output in the form of a feature vector. The feature vector of an embedding comprises an array of feature values, each representing a coordinate in a multidimensional vector space, and all together specify a point in said multidimensional vector space corresponding to the expression profile of the query perturbagen. The model 220 is further described with reference to Section IV.

The similarity scoring module 330 receives the embedding of the query perturbagen extracted by the model 220 and accesses embeddings for a set of known perturbagens from the known perturbagen store 310. The similarity scoring module determines 440 a similarity score between the embedding of the query perturbagen and the embedding of each known perturbagen. Accordingly, each similarity scores represent a measure of similarity between the query perturbagen input to the model 220 and a known perturbagen.

In one embodiment, the feature values of an embedding are numerical values such that a cosine similarity or Euclidean distance between two arrays of feature values for two perturbagens (either known or query) is considered a metric of functional similarity. Pairs of perturbagens corresponding to higher cosine similarities or lower Euclidean distances separating their respective embeddings may be considered more similar than pairs of perturbagens corresponding to lower cosine similarities or higher Euclidean distances.

The similarity scoring module 330 and ranks each of the similarity scores calculated from the embeddings such that similarity scores indicating greater functional similarity are ranked above similarity scores indicating lower functional similarity. In some embodiments, the similarity scoring module 330 identifies a set of candidate perturbagens based on the ranked list of similarity scores. Candidate perturbagens refer to known perturbagens which may be functionally/pharmacologically similar to the query perturbagens based on their effects on gene expression. In some embodiments, the similarity scoring module 330 selects one or more similarity scores within a range of ranks and determines 450 the known perturbagens associated with the selected similarity scores to be candidate perturbagens. In other embodiment, the similarity scoring module 330 may accessor receives a threshold rank stored in computer memory and selects similarity scores with rankings below that threshold rank. The known perturbagens associated with the selected similarity scores are determined to be candidate perturbagens.

In yet another embodiment, wherein higher similarity scores correspond to greater functional similarity, the similarity scoring module 330 accesses or receives a threshold similarity score stored within computer memory and selects known perturbagens corresponding to similarity scores above the threshold similarity score as candidate perturbagens. In yet another embodiment, wherein lower similarity scores correspond to greater functional similarity, the similarity scoring module 330 accesses or receives a threshold similarity score stored within computer memory and selects known perturbagens corresponding to similarity scores above the threshold similarity score as candidate perturbagens. In other embodiments, wherein the selection of the directionality (above or below) corresponds to a selection for known peturbagens sufficiently functionally similar to the query perturbagen, the similarity scoring module 330 does not identify any candidate perturbagens above or below a threshold similarity score, indicating that the query perturbagen does not share pharmacological properties with any known perturbagens of the known perturbagen store 310.

The functional property module 350 generates 460 an aggregate set of functional properties describing the pharmacological effects of each candidate perturbagen and assigns at least one property of the set to the query perturbagen. Functional properties describe the effect of a perturbagen on gene expression within a cell, for example upregulation of transcription for a specific gene or downregulation of transcription for a specific gene. In some embodiments, the functional property module 350 identifies 460 specific functional properties to the query perturbagen, while in others the module 260 assigns 460 the entire aggregate set of functional properties. The functional property module 350 may also store the set of functional properties associated with the query perturbagen.

In some embodiments the structural property module 360 provides additional insight into the query perturbagen by determining structural properties associated with the query perturbagen. For the candidate perturbagens, the structural module 270 accesses a Tanimoto coefficient describing the structural similarity between the query perturbagen and each candidate perturbagen and, for a more accurate assessment of structural similarities between the two perturbagens, averages the Tanimoto coefficient with the corresponding similarity score within the embedding extracted by the model 220. Based on the Tanimoto coefficient and embedding similarity score, the structural property module 360 determines a set of structural properties to be assigned to the query perturbagen. The structural property module 360 may also store the set of structural properties associated with the query perturbagen.

The evaluation module 370 evaluates the accuracy of the embeddings generated by the trained model 220. Example evaluations that may be carried out by the evaluation module 370 are discussed in Sections V and VI below.

### IV. Deep Neural Network Model

### IV.A Overview

The model 220 receives an expression profile for a query perturbagen as an input and encodes the expression profile into an embedding comprising an array of numbers, together describing a point in a multidimensional vector space corresponding to the query perturbagen. The model 220 uses the embedding of the query perturbagen, as well as embeddings of known perturbagens determined during training, to compute a set of similarity scores, each describing a likelihood that the query perturbagen is functionally similar to a known perturbagen. In embodiments in which the known perturbagen store 310 comprises expression data from the LINCS platform, the inputs to the model are vectors of standardized L1000 expression profiles. Because the L1000 platform measures gene expression data for 978 landmark genes, the vectors are 978-dimensional. Standardization may be performed per gene by subtracting the mean transcript count across all genes and dividing by the standard deviation of transcript count across all genes. Additionally, means and variances may be estimated over the entire training set.

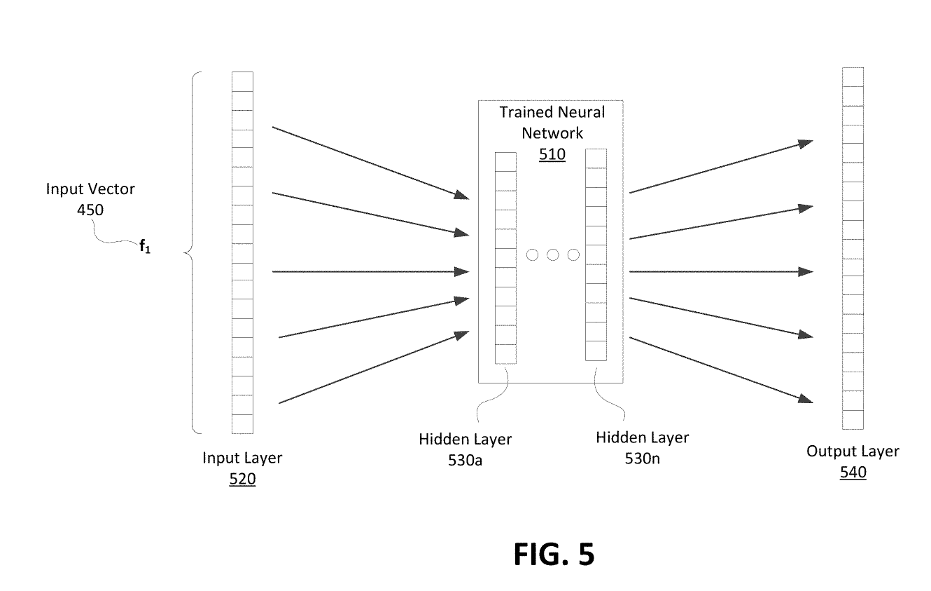

In one embodiment, the model 220 implements a deep neural network to extract embeddings from expression profiles associated with query perturbagens (or known perturbagens during training). FIG. 5 shows a diagram 500 of an exemplary neural network maintained by the model 220, according to one or more embodiments. The neural network 510 includes an input layer 520, one or more hidden layers 530-n, and an output layer 540. Each layer of the trained neural network 510 (i.e., the input layer 520, the output layer 540, and the hidden layers 530-n) comprises a set of neurons. Generally, neurons of a layer may provide input to another layer and may receive input from another layer. The output of a neuron is defined by an activation function that has an associated, trained weight or coefficient. Example activation functions include an identity function, a binary step function, a logistic function, a Tan H (hyperbolic tangent) function, an ArcTan (arctangent or inverse tangent) function, a rectilinear function, or any combination thereof. Generally, an activation function is any non-linear function capable of providing a smooth transition in the output of a neuron as the one or more input values of a neuron change. In various embodiments, the output of a neuron is associated with a set of instructions corresponding to the computation performed by the neuron. Here, the set of instructions corresponding to the plurality of neuron of the neural network may be executed by one or more computer processors.

In one embodiment, the input vector 550 is a vector representing the expression profile, with each element of the vector being the count of transcripts associated with one of the genes. The hidden layers 530a-n of the trained neural network 510 generate a numerical vector representation of an input vector also referred to as an embedding. The numerical vector is a representation of the input vector mapped to a latent space.

### IV.B. Example Neural Network Model

In the example implementation discussed in the examples of Section V, the neural network is deep, self-normalizing, and densely connected. The densely connected network may not use convolutions to train deep embedding networks and, in practice, observed no performance degradation during the training of networks with more than 100 layers. The neural network is more memory-efficient than conventional convolutional networks due to the lack of batch normalization. The final fully-connected layer computes the unnormalized embedding, followed by an L₂-normalization layer

$$\frac{1}{{‖ x ‖}_{2}} x ,$$

where x is the unnormalized embedding, to project the embedding on the unit hypersphere.

The network 410 is trained with a modified softmax cross-entropy loss over n classes, where each class represents an identity of a known perturbagen. For ease of notation, the following describes the loss function computed by the model 220 for a single query perturbagen. The loss for an entire set of query perturbagens is defined similarly. The model 220 implements the L₂-normalized weights with no bias term to obtain the cosines of the angles between the embedding of the expression profile and the class weight according to the equation:

$$cos {θ}_{i} = \frac{( {W}_{i}^{T} x )}{( {‖ {W}_{i} ‖}_{2} {‖ x ‖}_{2} )}$$

Where i represents the class index. The loss for a single training sample given θ={cos θᵢ}_{1≤i≤n}, where n is the number of classes (known perturbagens), is computed according to the equation:

$$- log \frac{exp ( ∝ ( cos {θ}_{i} - m ) )}{exp ( ∝ ( cos {θ}_{i} - m ) ) + {\sum }_{j \neq i} exp ( ∝ cos {θ}_{j} )}$$

Where i is the label (perturbagen identity), α>0 is a trainable scaling parameter and m>0 is a non-trainable margin parameter. During training, m is gradually increased from an initial value of 0, up to a maximum value. Inclusion of the margin forces the embeddings to be more discriminative, and serves as a regularizer. Embodiments in which the model 220 implements convolutional layers may use a similar margin.

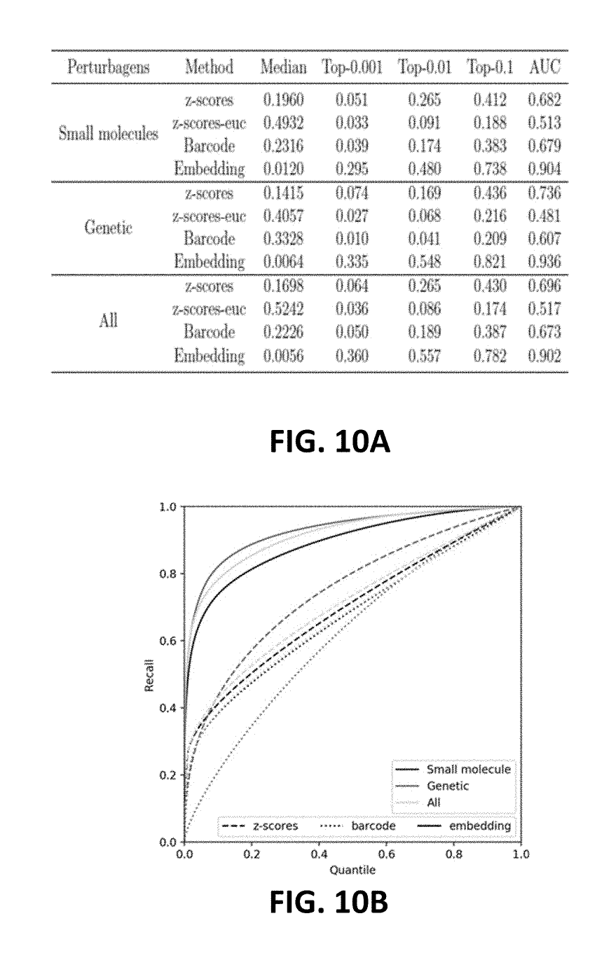

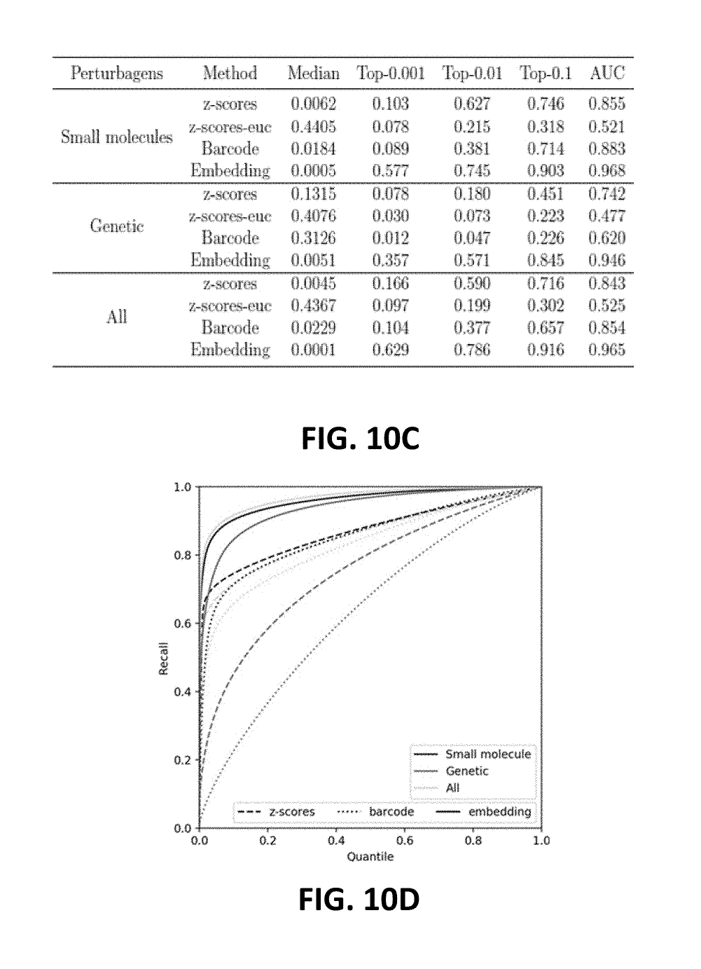

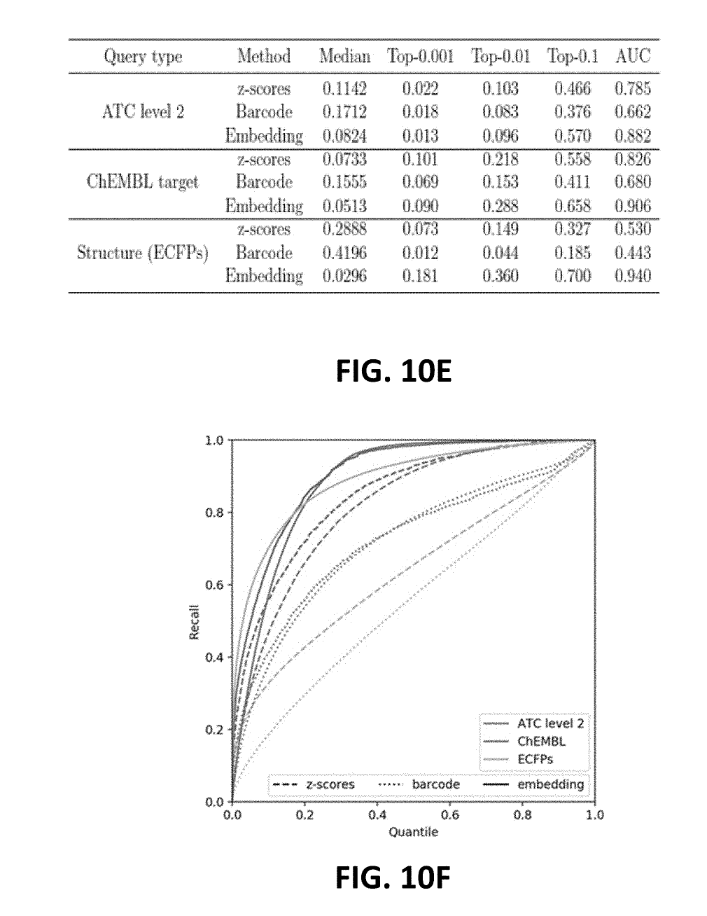

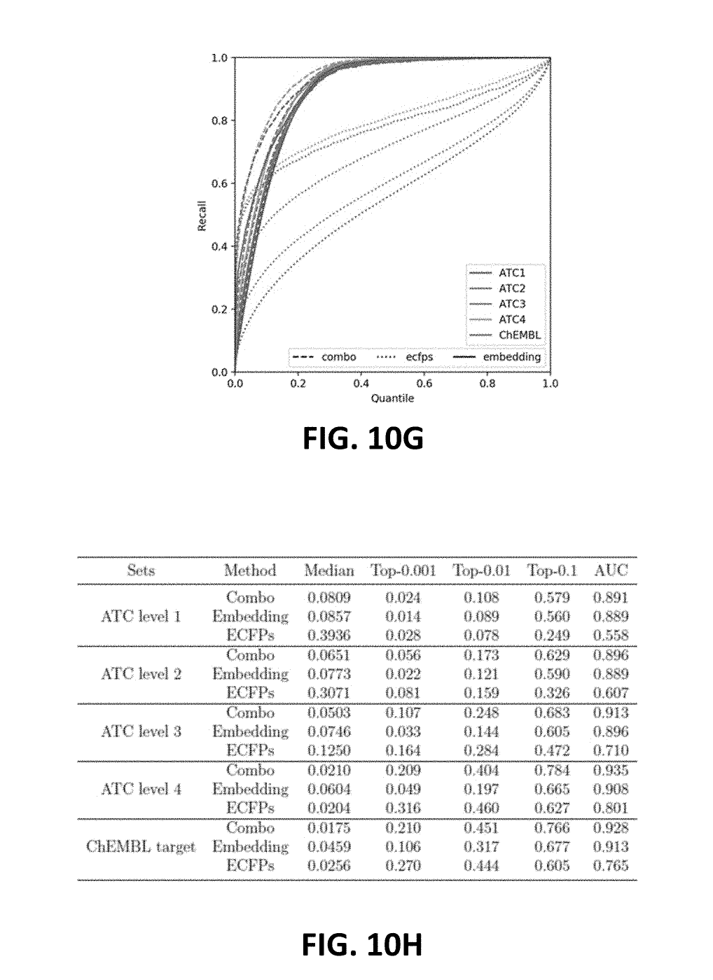

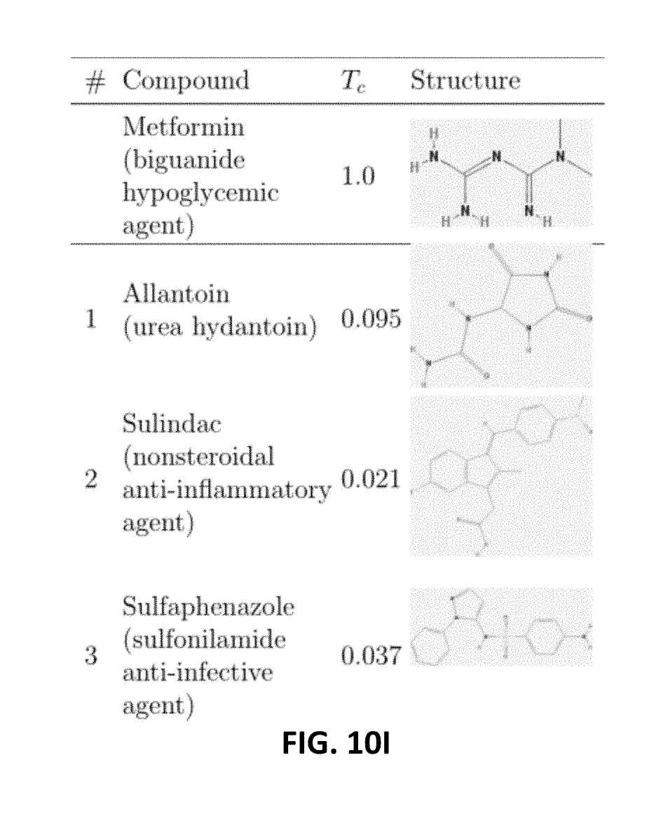

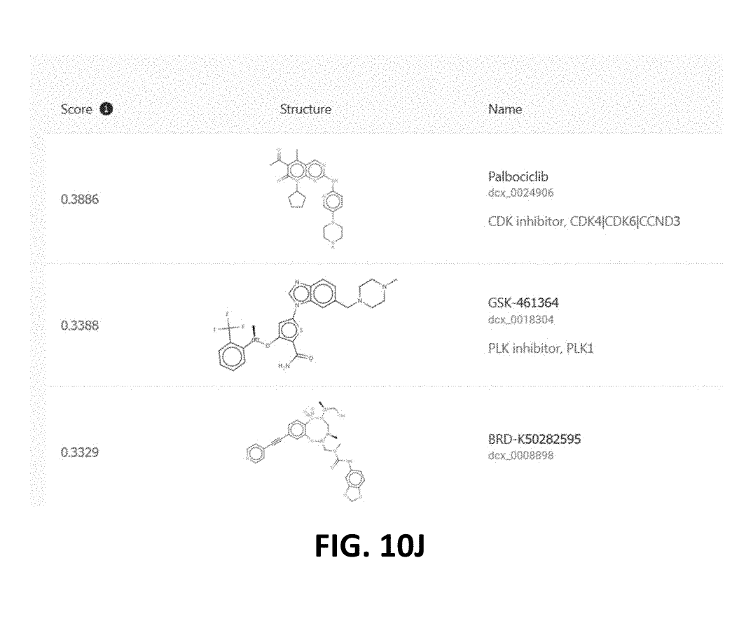

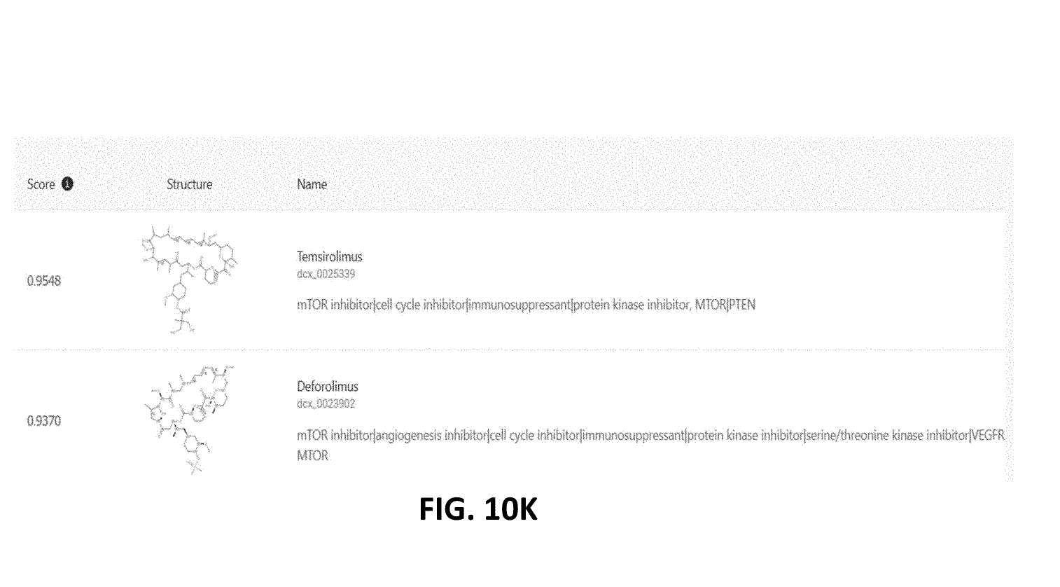

In one implementation evaluated for efficacy and accuracy, the trained neural network 410 has 64 hidden layers, growth rate of 16, and an embedding size of 32. The network is trained for 8000 steps with a batch size of 8192, adding Gaussian noise with standard deviation 0.3 to the input. The margin m is linearly increased at a rate of 0.0002 per step up to a maximum of 0.25. As will be discussed with reference to FIG. 10A-K, the predictive accuracy of a model employing such a network remains high over a wide range of values for all hyperparameters which prompted the conclusion that this example model structure may not be sensitive to hyperparameter values.

To maintain the main results reported with reference to FIG. 10A-K, several hyperparameters of the model 220 are established. The hyperparameters need not be optimized, but instead focused towards developing a robust network architecture that are not sensitive to hyperparameter choices. To achieve this robustness, the model is trained using any one or more of several factors: building blocks (SELU activations and densely connected architecture) that have proven effective at training very deep networks across many domains, data augmentation by injection of Gaussian noise into the standardized inputs, which diminishes the potential effects of overfitting, and regularization by the margin parameter, which like augmentation allows the training of large networks while reducing concerns of overfitting.

The hyperparameter values were set as follows. The margin parameter is set to 0.25, the noise standard deviation is set to 0.3, the embedding size is set to either 32, 64, or 128 with approximately 10-20 million parameter, and a growth rate of 16. In practice, it was determined that there was almost no difference between embedding sizes of 32 and 128 and embedding sizes of 32, 64, and 128 with nearly identical results. To emphasize generalization over separation in the metric space, the number of parameters may be decreased by an order of magnitude to about 1 million. The networks trained well at any depth. Because increasing the depth increases the training time, and the training loss changed very little with increases in depth, the depth value is set to 64. The training parameters of batch size, learning rate, and number of steps were determined empirically by examining the training loss during training.

For evaluation, rank distributions are aggregated over four splits to 80% training and 20% test perturbagens. First, the model was evaluated at embedding sizes between 4 and 128 in powers of 2. Since one training goal is to separate the perturbagens to clusters of similar perturbagens by pharmacological functionality, there may be an embedding size large enough to completely separate all the perturbagens in a space where distance is functionally meaningless. The performance of the model increased rapidly up to an embedding size of 32 and change very little beyond that.

Keeping the embedding size at 32, the network depth was evaluated. The number of parameters in the network is a function of the depth d and the growth rate k. The input size is 978 and each layer grows by k, so the number of parameters is equivalent to:

n(d,k)=Σᵢ₌₀^{d-1}k(978+ki)=978kd+1/2k²d(d−1).

To experiment with depth, the number of parameters N and for a given d chose the minimal k>0 such that n(d, k)≥N. With the number of parameters approximately fixed, performance improved with increasing depth up to 16 and beyond that point it remained almost constant up to 128, the deepest network that was trained. Subsequently, the depth was fixed to 16. For the number of parameters, performance improved with the number of parameters even above 1 million parameters, but the improvement beyond 400K parameters was small. Accordingly, evaluation of the model was performed with 1 million parameters corresponding to a growth rate of 45.

Training with a maximum value of the margin between 0.1 and 0.5 did not result in a variance of the performance. Similarly, adding no noise decreased the performance significantly, but performance was stable and optimal in the range between 0.3 and 0.6.

### IV.C. Embeddings Generated Based on Expression Profiles

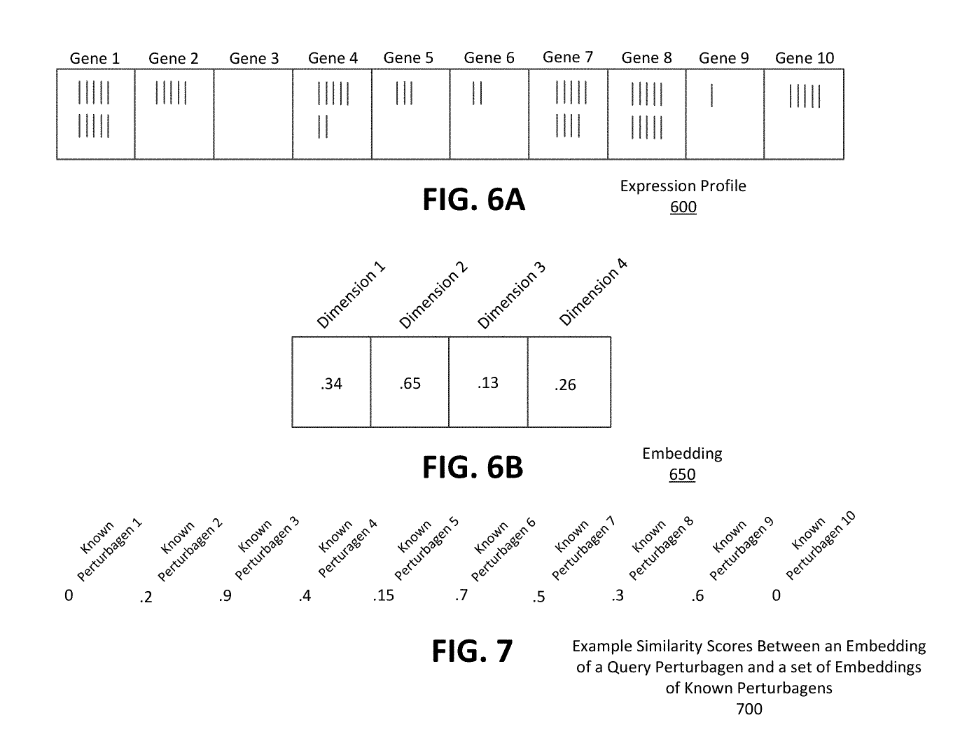

As introduced above, the model 220 generates embeddings based on an expression profile received for a perturbagen. As described above, expression profiles describe the transcription of genes within a cell, for example the number of transcriptomes (i.e., transcript counts) for each of a set of genes. FIG. 6A shows an exemplary expression profile of a cell affected by a perturbagen, according to one or more embodiments. The illustrated expression profile 600 describes gene expression data for 10 genes which are being transcribed at various rates. The transcription rates for each gene are represented by tallies describing the number of transcriptomes expressed in response to the introduction of a perturbagen. Specifically, the expression profile indicates that gene 1, gene 7, and gene 8 continue to be expressed at high rate compared to gene 3, gene 9, gene 6, and gene 5. The profile analysis module 120 compares the expression profile of the perturbagen-affected cell with expression profiles affected by known perturbagens to identify a set of candidate perturbagens functionally similar to the query perturbagen.

Also as introduced above, to determine a set of functional properties associated with a query perturbagens, an expression profile for a query perturbagen is input to the model 220 to generate an embedding of the expression profile. FIG. 6B shows an exemplary embedding, according to one or more embodiments. The model 220 extracts, from the expression profile, an embedding 650 in which a feature value is generated for each dimension of the embedding. FIG. 6B illustrates an example where the dimensionality of the embedding (four dimensions) is less than the dimensionality of the expression profile (ten genes).

### IV.D Computation of Similarity Scores

To determine functional properties of a query perturbagen, the similarity scoring module 330 identifies a set of candidate perturbagens with similar effects on cells as the query perturbagen. For example, if a query perturbagen upregulates the expression of genes A, B, and C, a known perturbagen which also upregulates the expression of genes A, B, and C will have a higher similarity score for the query perturbagen than a known perturbagen which downregulates the expression of genes A, B, and C.

A similarity score between two embeddings may be determined by computing the normalized dot product (also referred to as a cosine distance or cosine similarity) between the embedding of a first perturbagen (e.g., a query perturbagen) and the embedding of a known perturbagen (e.g., a known perturbagen).

FIG. 7 shows an exemplary set of similarity scores computed between an embedding of a query perturbagen and embeddings of a set of known perturbagens, according to one or more embodiments. The illustrated set of similarity scores 700 comprise similarity scores for 10 known perturbagens compared against a hypothetical query perturbagen, for example the query perturbagen associated with the expression profile 600. When the similarity score between two embeddings is calculated as the cosine distance, it is a numerical value within the inclusive range of −1 and 1. Accordingly, the similarity scores comparing the functional properties of each known perturbagen and a query perturbagen also range between −1 and 1. In one embodiment, similarity scores closer to 1 indicate a higher likelihood that the query perturbagen is functionally similar to a known perturbagen, whereas similarity scores closer to −1 indicate a lower likelihood that the two perturbagens are functionally similar. Therefore, with regards to the embedding 700, the query perturbagen is most functionally similar to known perturbagen 3 and candidate known 6, with similarity scores of 0.9 and 0.7, respectively. Additionally, the query perturbagen is least functionally similar to known perturbagen 1 and known perturbagen 10 which both have similarity scores of 0.

In another embodiment, the similarity score between two embeddings is calculated as the Euclidean distance, a numerical value greater than or equal to 0. Accordingly, the similarity scores comparing the functional properties of each known perturbagen and query perturbagen are greater than or equal to 0. Similarity scores closer to 0 may indicate a higher likelihood that the query perturbagen is functionally similar to a known perturbagen, whereas similarity scores farther from 0 may indicate a lower likelihood that the query perturbagen is functionally similar to a known perturbagen.

Continuing with the example from FIG. 7, the similarity scoring module 330 ranks the similarity scores for the ten known perturbagens and, in one embodiment, determines one or more of the highest ranked perturbagens to be candidate perturbagens which are functionally similar to the query perturbagen. For example, if the similarity scoring module 330 determines candidate perturbagens based on a comparison to a threshold similarity score of 0.55, known perturbagens 9, 6, and 3 would be candidate perturbagens. Alternatively, if the similarity scoring module 330 selects the four highest ranked perturbagens, known perturbagen 7 would also be a candidate perturbagen.

In some embodiments, similarity scores are based on binary classification, for example 0 and 1, where one binary label (e.g., “1”) indicates that that a known perturbagen is functionally similar to the query perturbagen and the other (e.g., “0”) indicates that a known perturbagen is not functionally similar. In such embodiments, the model may initially determine non-binary similarity scores for multiple known perturbagens and then assign a binary label to each of the similarity scores. Each label of the binary system may describe a range of similarity scores bifurcated by a threshold value (i.e., similarity scores above the threshold value indicate an acceptable likelihood that a known perturbagen is functionally similar to a candidate perturbagen whereas similarity scores below the threshold value do not). As a result, the similarity scoring module may determine that any known perturbagens assigned the label indicating an acceptable likelihood are candidate perturbagens.

Because the input to the model 220 is a single L1000 expression profile of a cell effected by a perturbagen, the output is an embedding for a single expression profile and candidate perturbagens are determined for that single expression profile. However, embeddings may also be determined for perturbagens corresponding to multiple expression profiles rather than for individual expression profiles. Such embeddings may be referred to as perturbagen-level embeddings. To generate a perturbagen level embedding, the module 320 may determine an average of all embeddings for expression profiles perturbed by that perturbagen. This may be calculated by averaging the features values of each feature of the embeddings associated with the same perturbagen (i.e. the nth feature value of the average embedding is calculated by determining the average of the nth feature values of all of the embeddings for expression profiles perturbed by that perturbagen). In other embodiments, aggregate measures other than simple averaging may be used to generate the perturbagen level embedding. As described above with profile-level embeddings, the similarity scoring module 330 receives the perturbagen-level embedding of a query perturbagen and determines a similarity score between the embedding of the query perturbagen and a perturbagen-level embedding of at least one known perturbagen. The similarity scoring module ranks the similarity scores and identifies a set of candidate perturbagens associated with similar functional properties.

A more thorough description of the computation of similarity scores is described as follows. Let {pᵢ}_{1≤i≤P} and {nⱼ}_{1≤j≤N} denote the similarity scores for positive and negative candidates, where P and N are the total numbers of positive and negative candidates for that specific query. Here, a positive group of candidates comprises known perturbagens sharing functional similarities with a query perturbagen, whereas the negative group of perturbagens comprises known perturbagens which do not share functional similarities with the query perturbagen. Perturbagens within the positive group are eligible to be accurately considered candidate perturbagens. In the embodiments described below, the evaluation is performed using profile-level analysis.

To perform the evaluation, the embedding of an expression profile of a query perturbagen is compared to the embedding of expression profiles of known perturbagens within the positive group, as well as to the embeddings of expression profiles of known perturbagens within the negative group. For each 1≤i≤P, the rank quantile of the similarity scores of the ith positive pᵢ within all negative scores is computed as follows:

$${q}_{i} = \frac{1}{N} {{\sum }^{N}}_{j = 1} \frac{1}{N} ( sgn ( {n}_{j} - {p}_{i} ) + 1 )$$

where sgn(x) is the sign function.

For the examples presented herein, the similarity scoring module 330 aggregates the quantiles qᵢ over all query perturbagens using the following metrics: the median rank, and the top-0.001, top-0.01, and top 0.1 recall. The term “recall” refers to the fraction of positives retrieved (i.e., the number of positives retrieved divided by the total number of positives). More specifically, the top-x recall r(x), defined for x∈[0, 1], indicates that the fraction of positives (i.e., known perturbagens in the positive grouped) ranked higher (lower quantile) than the x quantile:

$$r ( x ) = \frac{1}{Q} {{\sum }^{Q}}_{i = 1} \frac{1}{2} ( sgn ( x - {q}_{i} ) + 1 )$$

where Q is total number of positives over all queries and the vector q of the size Q is the concatenation of all the quantile vectors from the individual queries.

As referred to herein, ranks are referred to as “quantiles,” which describe a relative rank out of the number of negatives (i.e., known perturbagens in the negative group). For example, rank 689 of 10,000 is the 0.0689 quantile. Such an approach accounts for changes in the number of negatives between experiences, without assuming that negatives are drawn independently from an identical distribution and that the positive is expected to remain the same. As a result, rank 689 of 10,000 is considered the same as rank 6890 out of 100,000.

The model 220 assigns similarity scores using two approaches. The first method implements a recall by quantile. For each quantile q∈[0, 1] (x-axis), the recall r(x) is the y-axis value. As explained above, the recall is the fraction of query perturbagens in which the positive is ranked above q. For example, for q=0.05, the x-axis value is 0.05 and the fraction of queries in which the positive is ranked in the top 5% is the y-axis value. The first method evaluates a Receiver Operating Characteristic (ROC) curve, where the true positive rate (y-axis) is plotted against the false positive rate (x-axis).

More generally, similarities between perturbagens may be characterized according to three considerations: (1) shared pharmacological or therapeutic properties, (2) shared protein targets, and (3) structural similarity. Shared pharmacological or therapeutic properties which may be defined using anatomical therapeutic classifications (ATC). There are four relevant ATC levels, ranging from the most general level 1, which describes the main anatomical group, to the most specific level 4, which describes the chemical, therapeutic, and pharmacological subgroup. In some embodiments, ATC level 2 which describes the therapeutic subgroup is implemented by the model 220 because it adds additional information to evaluation of functional similarity beyond the biological protein targets defined by ChEMBL. Other embodiments, implement all four ATC levels.

Shared biological protein targets may be defined using ChEMBL classifications based on experiments performed in wet lab simulation in which the simulation applies the query peturbagen to a cell. The simulation measures transcription counts for a set of genes within the cell to characterize the change in gene expression caused by the perturbagen and determines one or more biological targets within the cell affected by the query perturbagen. The correlation between the affected biological targets and the change in gene expression may be stored within the profile analysis module 120.

Structural similarities are defined by Tanimoto coefficients for ECFPs and MACCS keys. In one embodiment involving ECFPs, known perturbagens with Tanimoto coefficients above 0.6 were considered to be structurally similar to a known perturbagen and known perturbagens with Tanimoto coefficients below 0.1 were considered to not be structurally similar. In another embodiment involving MACCS keys, known perturbagens with Tanimoto coefficients above 0.9 were considered to be structurally similar to a known perturbagen and known perturbagens with Tanimoto coefficients below 0.5 were considered to not be structurally similar.

In one specification “combination” embodiment, the model 220 complements determinations of functional properties assigned by the functional property module 350 through the use of the neural network model with structural similarities between a known perturbagen and a query perturbagen as provided by Tanimoto coefficients. As discussed below with reference to Example IV, this leads to a more accurate evaluation of the overall similarity between a query and candidate perturbagens than from either factor (e.g., structural or functional similarity) alone. In one embodiment, the structural property module 360 computes an unweighted average of the similarity score calculated by the similarity scoring module 330 (which represents functional similarity) and the EFCPs Tanimoto coefficient. In alternate embodiments, the similarity score calculated from an embedding and the EFCPs Tanimoto coefficient may be combined using a different computation.

### IV.E. Model Training

In an iteration, the neural network is trained using only the labeled profiles from the known perturbagen store 310. At the end of each iteration, the trained neural network runs a forward pass on the entire dataset to generate feature vectors representing sample expression data at a particular layer. These profiles are then labeled, and are added to the labeled sample set, which is provided as input data for the next training iteration.

FIG. 8 shows an exemplary training data set of known perturbagens, according to one or more embodiments. As illustrated in FIG. 8, the training data store 310 comprises five known perturbagens (e.g., known perturbagen 1, known perturbagen 2, known perturbagen 3, known perturbagen 4, and known perturbagen 5). Each known perturbagen is associated with multiple expression profiles, for example known perturbagen 1 is labeled as associated with expression profile 1A and expression profile 1B.

During training, the labeled expression profiles are input to the model 220 to extract embeddings. Similarity scores are computed between the extracted embeddings associated with a same known perturbagen. Additional similarity scores may be generated between the extracted embeddings for expression profiles associated with different known perturbagens. The model is generally trained such that the similarity scores between embeddings of labeled expression profiles from the same known perturbagen are higher, and similarity scores between embeddings of labeled expression profiles from different known perturbagen are lower. For example, an embedding extracted from expression profile 1A, when compared to embeddings extracted from expression profiles 1B and 1C, should result in a similarity score indicating a comparatively high level of similarity. Comparatively, a similarity score computed between the embedding extracted from expression profile 1A and the embedding extracted from expression profile 2D, or any other expression profile associated with known perturbagens 2, 3, 4, and 5, should indicate a comparatively low level of similarity,

### V. Internal Verification of the Model

Because the profile analysis module 120 ranks known perturbagens in silico based on their functional similarity to a specific query perturbagen, the determination based on the extracted embedding is evaluated for an improvement in accuracy over conventional in vitro or in vivo methods. The model 220 is evaluated for its generalization ability using cross-validation at the perturbagen level.

To evaluate the model, the evaluation module 370 compares the similarity scores determined between a single query perturbagen and many known perturbagens from set which including both a positive group and a negative group, as defined above in Section IV.D.

In one embodiment, the module 220 is evaluated using individual expression profile-level embeddings for query perturbagens, known perturbagens, and candidate perturbagens. For a first evaluation, the evaluation module 370 tests the performance of the model based on a set of profiles associated with the same perturbagen. Returning to FIG. 8, for example, the model may be evaluated using a data set comprised of the five expression profiles associated with known perturbagen 4.

FIG. 9 shows a flow chart of the process for internally evaluating the performance of the model, according to one or more embodiments. Prior to the training or evaluation of the model, the profile analysis module 120 divides 910 the contents of the known perturbagen datastore into a hold-out dataset and a training dataset. The model 220 is trained using the training dataset without being exposed to the hold-out dataset.

As a result, the evaluation model 370 tests the model 220 using the hold-out dataset without having biased the model 220. The evaluation module 370 selects an expression profile for a query perturbagen from the hold-out dataset and divides 920 the holdout group into a positive dataset/group and a negative dataset/group. Returning to the example in FIG. 8, if the evaluation module 370 were to select expression profile 4A, expression profiles 4B, 4C, 4D, and 4E would comprise the positive group and the remaining expression profiles for known perturbagens 1, 2, 3, and 5 would comprise the negative group. In one embodiment, the hold-out dataset comprises a random subset of 1000 known perturbagen profiles, however it is to be understood that in practice, the known perturbagen store 310 comprises an expansive group of expression profiles, for example the entirety of the 1.5 million perturbagens within the LINCS dataset.

The model 220 extracts 930 an embedding from each known perturbagen of the hold out group. Using the embedding of the query perturbagen to compute similarity scores, the evaluation module determines 940 similarity scores between the extracted embeddings and the embeddings of expression profiles within both the positive group and the negative group. Because expression profiles within the hold-out dataset are labeled with their corresponding known perturbagen, the model, at optimal performance, generates an embedding which confirms 950 the corresponding known perturbagens for the positive hold out group as the candidate perturbagens.

In one embodiment, the confirmation is determined by reviewing the rankings of known perturbagens generated by the similarity scoring module 330. If all of the expression profiles in the positive group correspond to higher rankings than expression profiles in the negative group, the model is evaluated to be performing optimally. Since the similarity scoring module 330 ranks known perturbagens based on the similarity scores determined by the model 220, by confirming that the ranked list prioritizes expression profiles within the positive group as candidate perturbagens over the expression profiles within the negative group, the evaluation module 370 verifies the performance of the model 220. The evaluation process may be repeated using different expression profiles stored within the hold-out dataset.

In additional embodiments, the evaluation module 370 verifies the performance of the model using a more specific set of expression profiles, namely biological replicates. For general profile-level queries, the positive group is comprised of expression profiles from the same known perturbagen as the query perturbagen and the negative group is comprised of profiles in which a different perturbagen was applied. By contrast, for profile-level queries of biological replicates, the positive group comprises expression profiles sharing a combination of the same known perturbagen, a cell line, a dosage, and a time at which the dosage was applied. The negative group comprises expression profiles of the hold-out group which do not meet those criteria.

Because functional labels are not used during training of the model 220, there is no concern of overfitting; however, training and evaluating the network on the same samples could potentially lead to worse results when evaluating functional similarity. For example, if two different perturbagens in the data have nearly identical functional outcomes, their embeddings should be nearly identical, but the training objective will push their embeddings apart, resulting in an underestimation of their similarity. By contrast, if at least one of the perturbagens is not included in the training set, the similarity would not be underestimated and could be very close to 1. However, because underestimating the similarity affects all perturbagen pairs, it may not negatively impact the ranking of perturbagens.

To determine whether evaluating the embeddings directly on the training set negatively impacts the ranking of perturbagens by similarity, the similarity between perturbagens was computed using a holdout method. For this purpose, the entire set of perturbagens was split 41 times into a training set and test set. The splits were generated such that each pair of perturbagens appeared at least once in the same test set. The neural network was trained 41 times, once on each training set, and the embeddings for the corresponding test set samples were computed. The similarity between two perturbagens was computed as the average similarity between them over all test sets in which both appeared. Within each test set, perturbagen similarity was computed as the average dot product of sample embeddings, as before. Finally, the ranking summary statistics and AUC were using ATC levels 1-4 and ChEMBL as queries.

The results obtained using the holdout similarity method were similar to those obtained using the direct training method, with a small difference in favor of the latter (Table S8 and Figures S15 and S16). The similarity ranks for each perturbagen were compared using Kendall's tau rank distance. 90% of the distances were below 0.32, with a mean distance of 0.2615. In practice, similarity in embedding space is used to find perturbagens that are functionally similar to a query perturbagen, so only the top ranks are important. The joint distribution of similarity quantiles over all pairs of perturbagens using the direct training method and the holdout method were visualized. For the top 10% similarities, the correspondence was accurate.

### VI. External Verification of the Model

While the internal evaluation, described above in Section V., evaluated the ability of the model 220, the similarity scoring module 330, and the functional property module 350 to determine functional similarities between a query perturbagen and known perturbagens, the evaluation module 370 can conduct an additional evaluation to verify the ability of the model 330 to identify structurally similar compounds. As described above with reference to the internal evaluation of the model, the same hold-out group is used to evaluate structural similarities. Within the hold-out group, perturbagens within the positive group and the negative group are determined by the structural similarity of known perturbagens to the query perturbagen. Structural similarity is defined as the Tanimoto coefficient for extended connectivity fingerprints. In one embodiment, the positive group for a query perturbagen comprises known perturbagens with a Tanimoto coefficient above 0.6 and the negative group for a query perturbagen comprise known perturbagens with a Tanimoto coefficient below 0.1. In another embodiment, the hold-out group is divided using MACCS. The performance of the model 220 is evaluated separately for two disjoint groups of perturbagens: a first group comprising small molecules and a second group comprising direct manipulations of target gene expression through CRISPR or expression of shRNA or cDNA.

In one embodiment, the module 370 compares the functional similarities and structural similarities determined based on the embedding generated by the model 220 with two sets of baseline data accessed from the baseline data store 380: the z-scores of the 978 landmark genes, hereafter referred to as “z-scores,” and perturbation barcode encoding of L1000 profiles, hereafter referred to as “barcodes.”

When compared to the z-score, the evaluation module 330 confirmed that the model 220 performed better than z-score approach. The similarity score between a query perturbagen and a known perturbagen was compared to the corresponding z-score and the Euclidean distance between a query perturbagen and a known perturbagen was compared to the barcodes. A comparison of structural similarities was performed using ECFPs and MACCs keys with a Tanimoto coefficient measure of similarity.

### VI.A Example I

FIG. 10A is a table describing the performance of embeddings and baselines for queries of the profiles of the same perturbagen, by perturbagen group, according to an embodiment. FIG. 10B is a graph of the performance of embeddings and baselines for queries of the profiles from the same perturbagen, by perturbagen group, according to an embodiment.

For all perturbagen groups, the embeddings exhibited improvements over the baseline methods. Performance of the model on genetic manipulations is slightly better than on small molecules, but even when the evaluation was restricted to small molecules, the embeddings ranked the positive within the top 1% in almost half (48%) of the queries, and the AUC was above 0.9. These results suggest that the embeddings effectively represent the effects of the perturbagen with some invariance to other sources of variation, including the variance between biological replicates, as well as between cell lines, doses, post-treatment measurement delays, and other factors. Furthermore, because the evaluation only included perturbagens entirely held out during training, these results demonstrate that the embeddings generalized well to perturbagens not included in the training set.

### VI.B Example II

FIG. 10C is a table describing the performance of embeddings and baselines for queries of the profiles of the same set of biological replicates, by perturbagen group, according to an embodiment. For z-scores, results using Euclidean distances are reported. FIG. 10D is a graph of the performance of embeddings and baselines for queries of the profiles from the same set of biological replicates, by perturbagen group, according to an embodiment.

In this evaluation, positives were profiles that were biological replicates of the query perturbagen and negatives were select from among the profiles that were not. Both the embeddings and baseline methods performed better on this evaluation, but the embeddings still performed better than the baselines. The difference in performance between the biological replicate queries and the same-perturbagen queries was larger for the baselines than the embeddings. This may also reflect a level of invariance to sources of variation other than the perturbagen in the learned embedding space.

### VI.C Example III

FIG. 10E is a table describing the performance of embedding and baselines on queries of similar therapeutic targets, protein targets, and molecular structure, according to one embodiment. FIG. 10F is a graph of the performance of embedding and baselines on queries of similar therapeutic targets, protein targets, and molecular structure, according to one embodiment.

The embedding performed better than the baselines for all query types. The gap between the embeddings and baselines was largest for queries of structural similarity. Structurally similar compounds (the positives for each query) tend to have correlated expression profiles, but the correlations are weak. One possible explanation for this result is that the embedding is trained to cluster together profiles corresponding to the same compound, which is equivalent to identity of chemical structure. The greater similarities in embedding space between structurally similar compounds relative to structurally dissimilar compounds demonstrate good generalization of the training objective.

### VI.D Example IV

To determine to what extent embeddings generated by the model 220 complement measurements of structural similarity, the ability of structural similarity alone to identify drugs with the same known protein target or ATC classification was evaluated. Results are presented using ECFPs. FIG. 10G is a graph of the performance of structural similarity (ECFPs Tanimoto coefficient), embedding, and a combination of both on functional similarity queries based on ATC levels 1-4 and ChEMBL protein targets. FIG. 10H is a table of the performance of structural similarity (ECFPs Tanimoto coefficient), embedding and a combination of both on a functional similarity queries based on ATC levels 1-4 and ChEMBL protein targets. Structural similarity performed best on the lowest level sets, ATC level 4 (chemical/therapeutic/pharmacological subgroup) and ChEMBL protein targets, but its performance degraded rapidly when moving to more general ATC classifications, and for ATC levels 1 and 2 (anatomical/therapeutic subgroups), it was not much better than chance.

Even for ATC level 4 and ChEMBL targets, where structural similarity performed at its best, recall was excellent at the top ranks (top-0.001 recall of 0.316 for ATC level 4 and 0.27 for ChEMBL targets) but increased very slowly below the top 0.1 ranks. The distribution of ranks below the top 0.1 was very close to chance. In comparison with structural similarity, recall using our trained embeddings was not as high at the top ranks. However, with the exception of the top ranks, the embeddings performed better and addressed both of the structural similarity shortcomings described above. First, the performance degradation was very small at higher levels of classification, with good performance even at ATC level 1: median rank 0.086 and AUC of 0.889 (Table 5). Second, embedding recall kept increasing rapidly beyond the few top ranks, outperforming structural similarity starting at quantiles 0.0647 for ChEMBL, and 0.0867, 0.0544, 0.0202 and 0.0057 for ATC levels 4, 3, 2 and 1 respectively. These results indicate that the strengths of structural similarity and gene expression embedding could be combined, as discussed above.

The combination of embedding similarity score with EFCPs Tanimoto coefficient outperformed both structural similarity and the embeddings, demonstrating that the embedding generated by the model 220 constructively complements structural similarity, and that a combination of the two is more effective than either similarity alone.

### VI.E Example V

FIG. 10I shows a ranked list of known perturbagens corresponding to chemical compound treatments associated with a query perturbagen—the compound metformin, according to one or more embodiments. Additionally, FIG. 10I shows the Tanimoto coefficient representing structural similarity between the structure of metformin and the structure of each of the indicated known. In embodiments in which the similarity scoring module 330 implements a threshold rank of 1, the only known perturbagen which is identified by the model as a candidate perturbagen functionally similar to metformin is allantoin.

Metformin is an FDA-approved drug for diabetes which is known to lower glucose levels. Metformin affects gene expression in cells, in part, by mediating through its direct inhibitory effect upon imidazole I-2 receptors. The candidate perturbagen, allantoin, has similar pharmacological properties, in that allantoin lowers glucose levels, which is, in part, mediated through its direct inhibitory effect upon imidazole I-2 receptors. Additionally, given their Tanimoto coefficient of 0.095 it is understood that metformin and allantoin are not structurally similar.

The identification of allantoin as the candidate perturbagen with the greatest functional similarity to metformin according to the processes described herein, in combination with known wet lab experimental results demonstrating pharmacological similarities between metformin and allantoin, confirms that the model 220 is capable of accurately identifying structurally dissimilar perturbagens with pharmacological similarities to the query perturbagen. Accordingly, the model possesses utility for drug repurposing.

Additionally, if the mechanism of action of metformin were unknown, using a similarity rank threshold of 1 and assigning the therapeutic target of allantoin to be imidazole I-2 receptors determined from experimental data, the model 220 would have correctly inferred that the mechanism of action of metformin includes inhibition of the imidazole I-2 receptors. Accordingly, the model 220 possesses utility for identification of a previously unknown mode of action, such as a determination of the molecular target of a query perturbagen upon the basis of known molecular targets of known perturbagens which exceed a given similarity rank threshold.

### VI.F Example VI

FIG. 10J shows a ranked list of known perturbagens corresponding to chemical compound treatments associated with a query perturbagen—the knockdown of the gene CDK4. The illustrated list is ordered from greatest to least similar as measured by the cosine distance between the embedding of the query perturbagen and the embedding of each known perturbagen, according to an embodiment. Using a similarity threshold rank of 1, the only known perturbagen identified as functionally similar to the CDK4 knockout is the compound palbociclib. Palbociclib is a compound which exerts a pharmacological effect through inhibition of the genes CDK4 and CDK6.

Were the mechanism of action of palbociclib not known, using a similarity threshold rank of 1 and assigning the therapeutic target of palbociclib upon the basis of known perturbagens corresponding to gene knockouts or overexpressions passing the aforementioned similarity rank threshold, the model 220 would have correctly inferred that palbociclib exerts an inhibitory effect upon CDK4. This demonstrates that the model 220 has utility for inference of mechanism of pharmacological action of uncharacterized compounds.

### VI.G Example VII

FIG. 10K shows a ranked list of the known perturbagens corresponding to chemical compound treatments associated with a query perturbagen—the compound sirolimus. The illustrated list is ordered from greatest to least similar as measured by the cosine distance between the embedding of the query perturbagen and the embedding of each known perturbagen, according to one or more embodiments. Using a similarity threshold rank of 2, the only known perturbagens identified as functionally similar to sirolimus are the compounds temsirolimus and deforolimus.

Temsirolimus and deforolimus are both structurally and pharmacologically similar to sirolimus, as their chemical structures were designed upon the basis of the chemical structure of sirolimus and for the purpose of inhibiting mTOR, the molecular target of sirolimus.

This demonstrates that the model 220 identifies structures similar to sirolimus, or which use the structure of sirolimus as an initial scaffold for further optimization, as correctly possessing functional and therefore pharmacological similarity. Accordingly, the model 220 is capable of learning structure-function relationships. The model 220 is demonstrably capable of performing such structural inferences without being trained on structural information (i.e. the structures of the known perturbagens and of the query perturbagen were not used in the generation of their embeddings).

### VI.D Supplementary Data

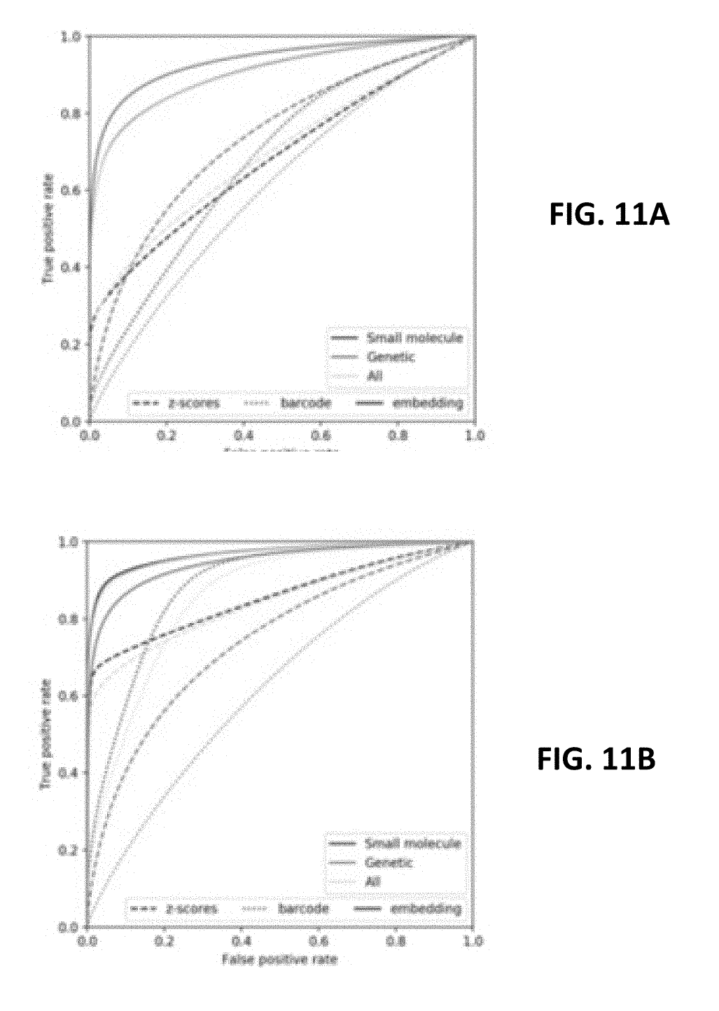

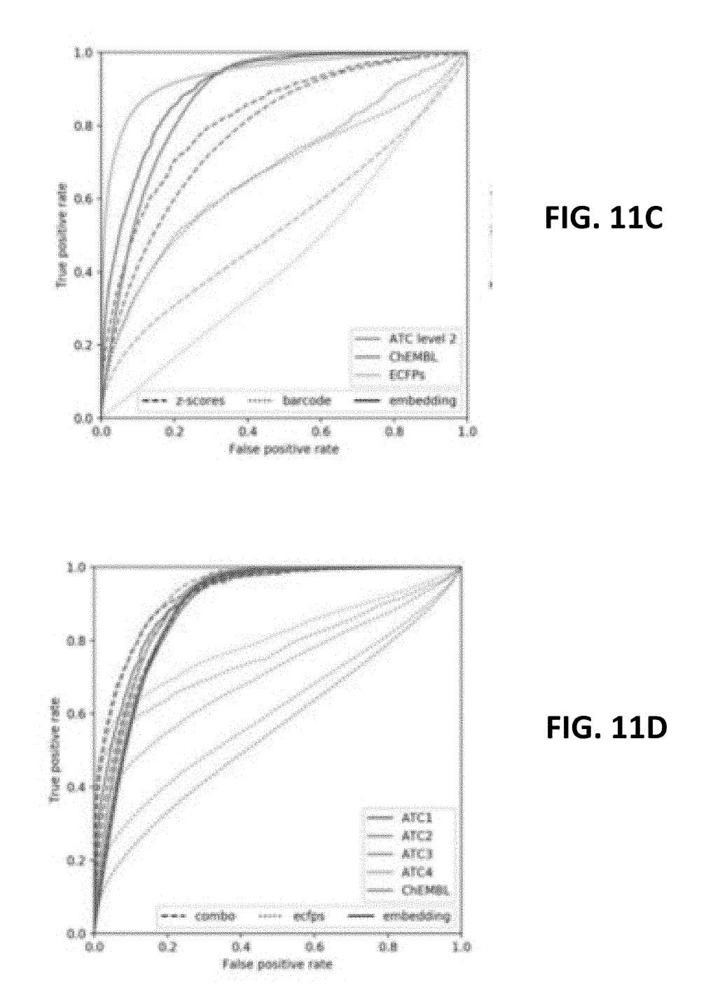

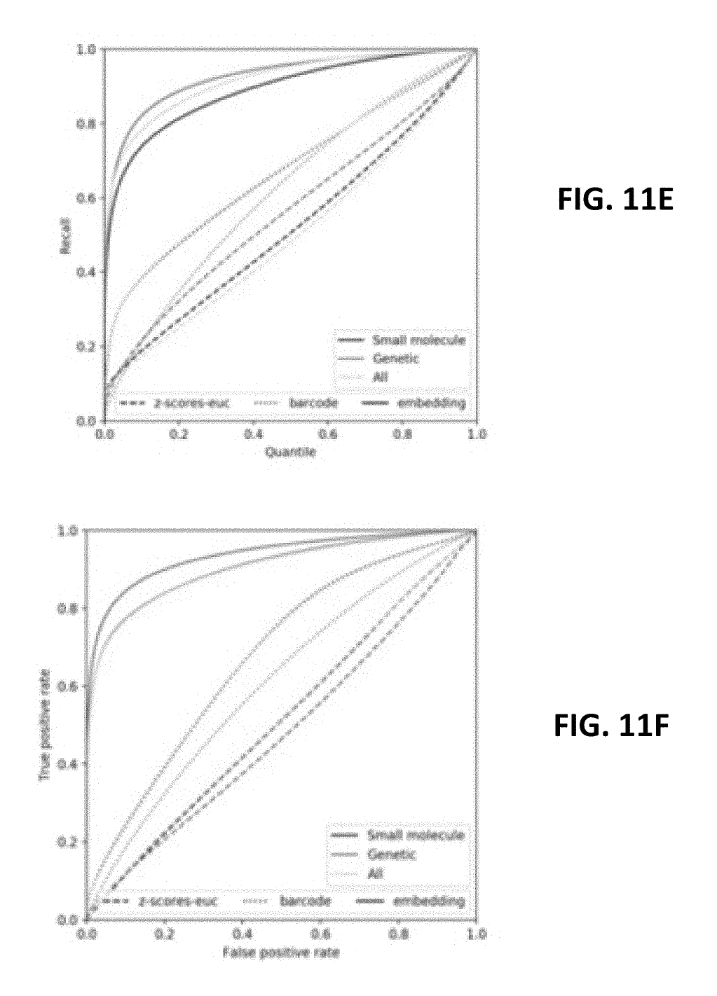

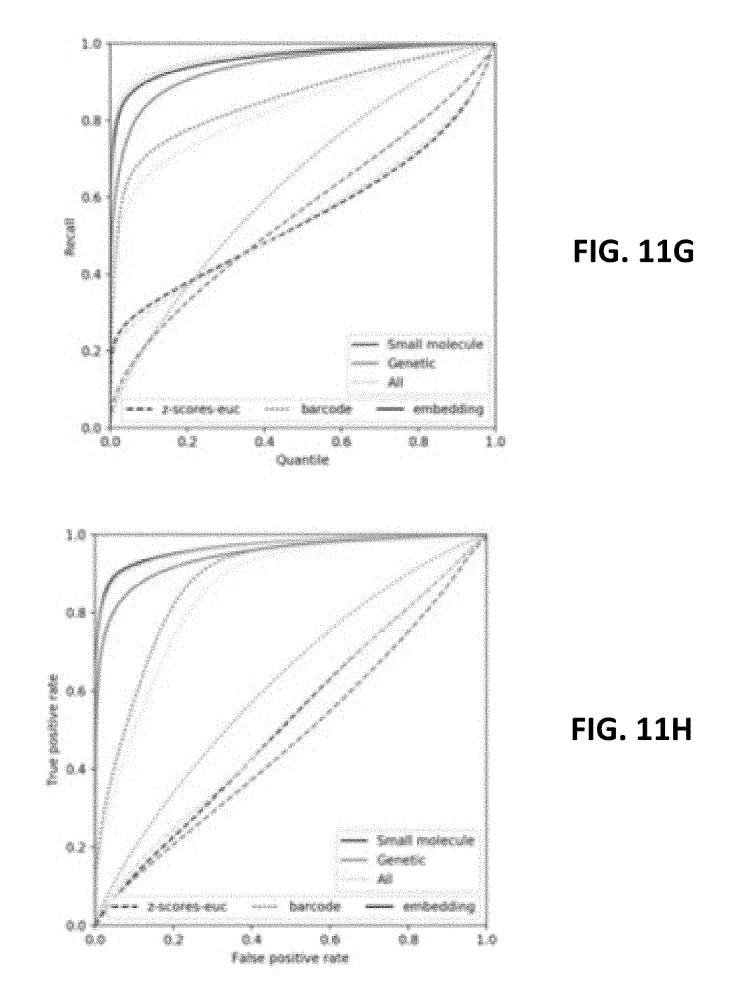

FIG. 11A is a graph of ROC curves of an embedding and baselines for queries for profiles from the same perturbagen, by perturbagen group.

FIG. 11B is a graph of ROC curves of embeddings and baselines for queries for profiles form the same set of biological replicates, by perturbagen group.

FIG. 11C is a graph of ROC curves of embeddings and baselines for queries of similar therapeutic targets, protein targets, and molecular structure.

FIG. 11D is a graph of ROC curves for structural similarity (ECFPs Tanimoto coefficient), embedding and a combination of both on functional similarity queries based on ATC levels 1-4 and ChEMBL protein targets.

FIG. 11E is a graph of recall by quantile for embedding and baselines on queries for profiles from the same perturbagen, by perturbagen group.

FIG. 11F is a graph of ROC curves for embedding and baselines on queries for profiles from the same set of biological replicates, by perturbagen group.

FIG. 11G is a graph of recall by quantile for embedding and baselines on queries for profiles from the same set of biological replicates, by perturbagen group.

FIG. 11H is a graph of ROC curves for embedding and baselines on queries for profiles from the same set of biological replicates, by perturbagen group.

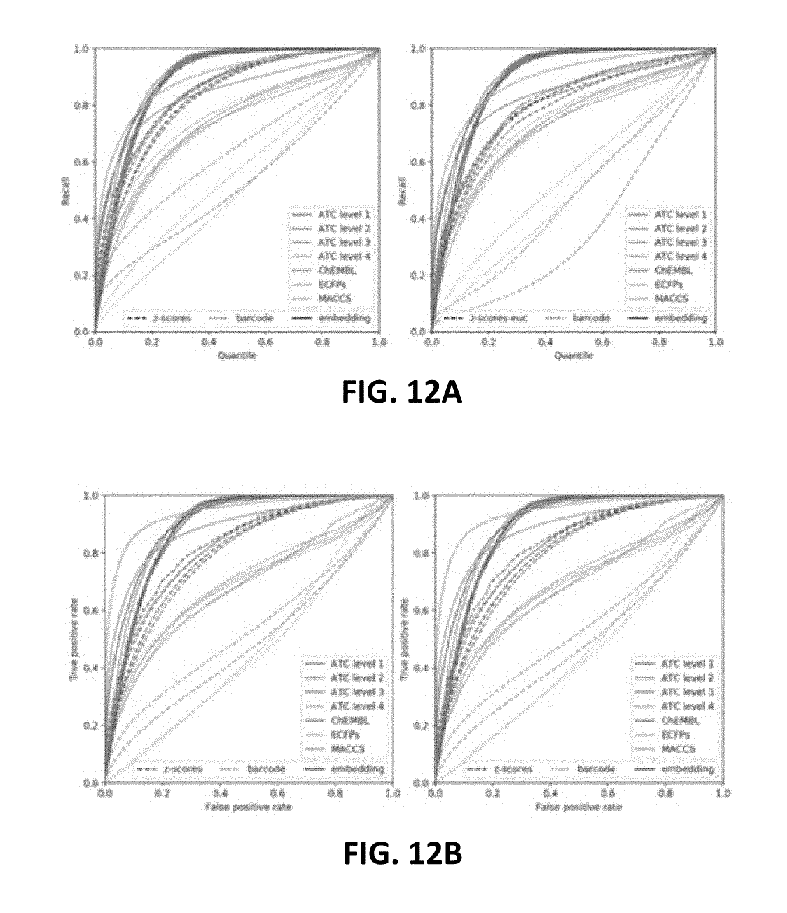

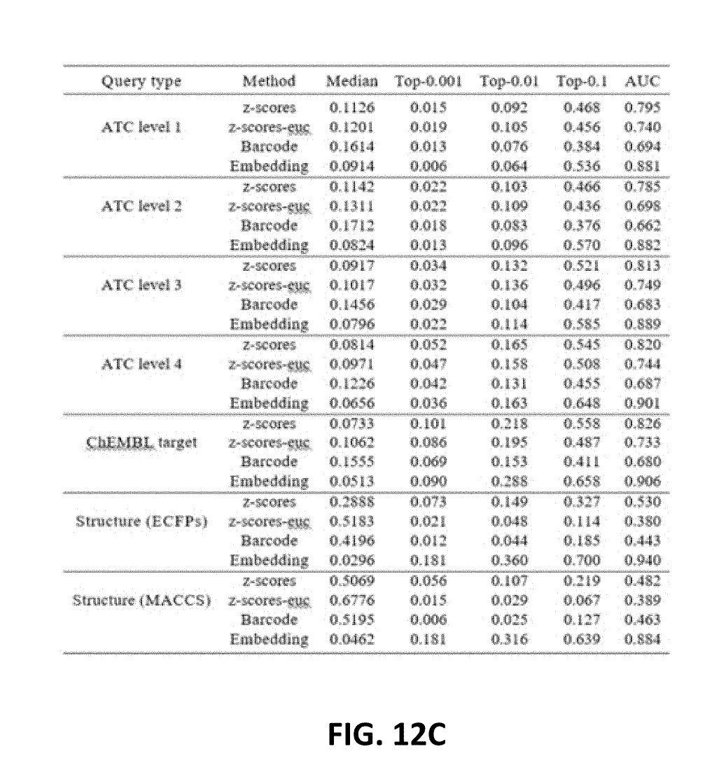

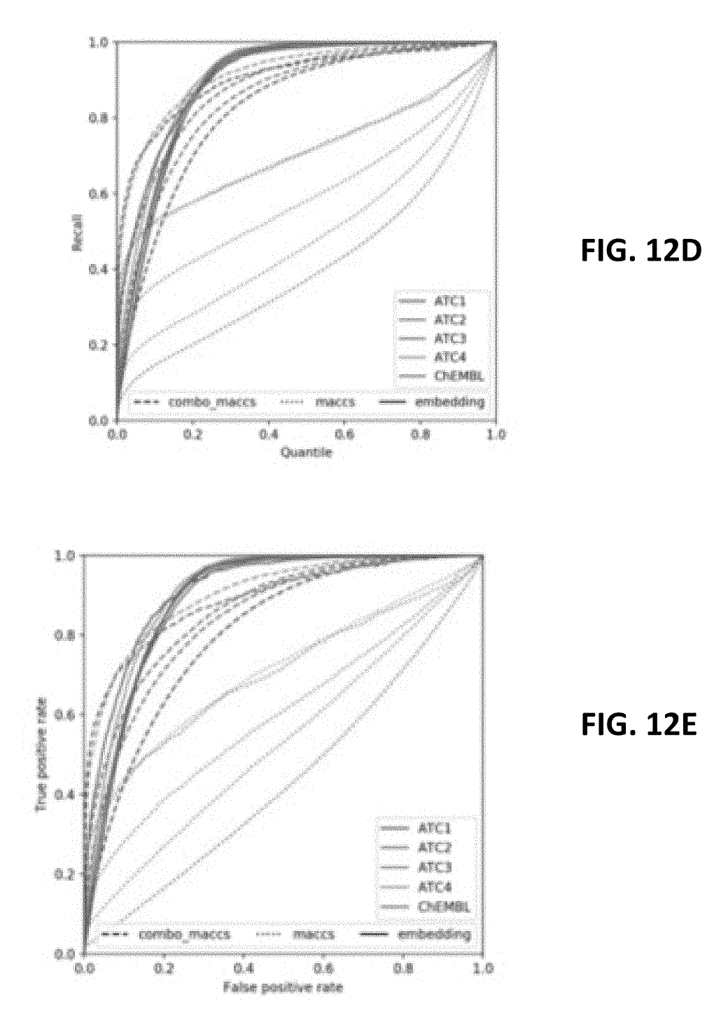

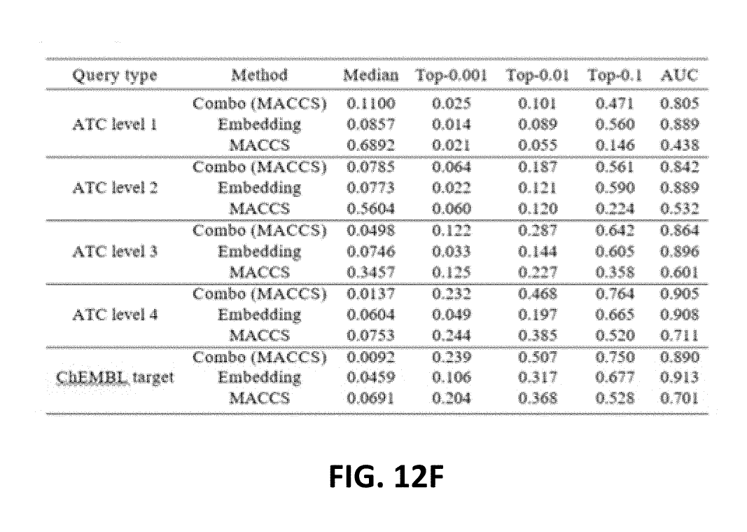

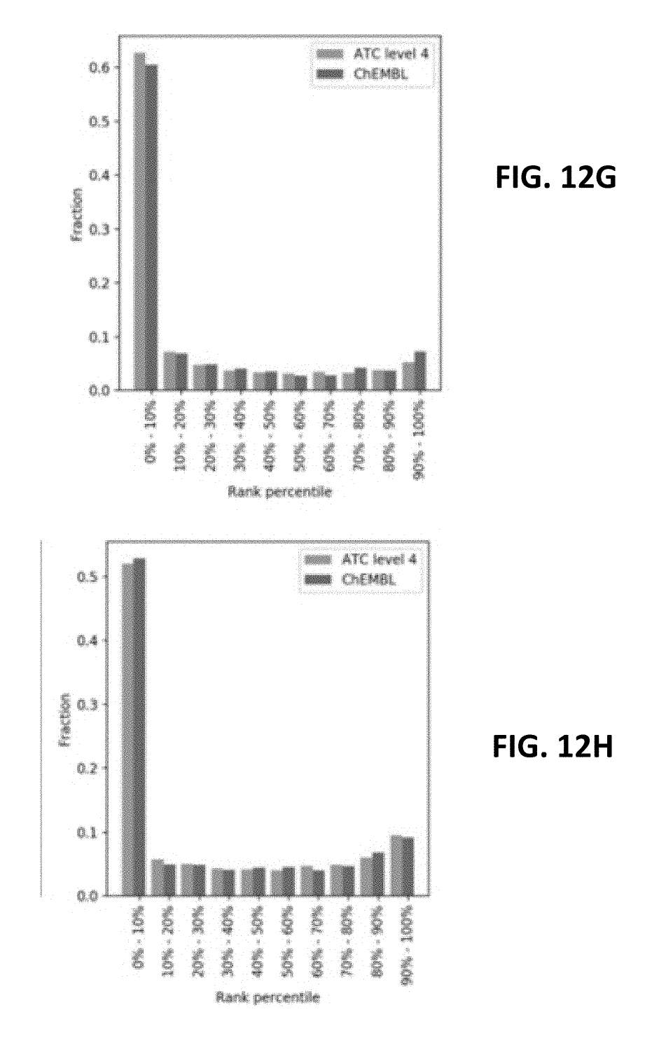

FIG. 12A is a graph of recall by quantile for embedding and baselines on queries of similar therapeutic targets (ATC levels 1-4), protein targets (ChEMBL) and molecular structure (defined by ECFPs and MACCS). For z-scores, cosine (left) or Euclidean (right) distance was used.

FIG. 12B is a graph of ROC curves for embedding and baselines on queries of similar therapeutic targets (ATC levels 1-4), protein targets (ChEMBL) and molecular structure (defined by ECFPs and MACCS). For z-scores, cosine (left) or Euclidean (right) distance was used.

FIG. 12C is a table of the performance of embedding and baselines on queries of similar therapeutic targets, protein targets and molecular structure.

FIG. 12D is a graph of recall by quantile for structural similarity (MACCS Tanimoto coefficient), embedding and a combination of both on functional similarity queries based on ATC levels 1-4 and ChEMBL protein targets.

FIG. 12E is a graph of ROC curves for structural similarity (MACCS Tanimoto coefficient) of embedding and a combination of both on functional similarity queries based on ATC levels 1-4 and ChEMBL protein targets.

FIG. 12F is a table of performance of embedding and baselines on queries of similar therapeutic targets, protein targets and molecular structure.

FIG. 12G is a histogram of rank quantiles for queries based on sets from ATC level 4 and ChEMBL, using ECFPs Tanimoto coefficient for similarity.

FIG. 12H is a histogram of rank quantiles for queries based on sets from ATC level 4 and ChEMBL, using MACCS Tanimoto coefficient for similarity.

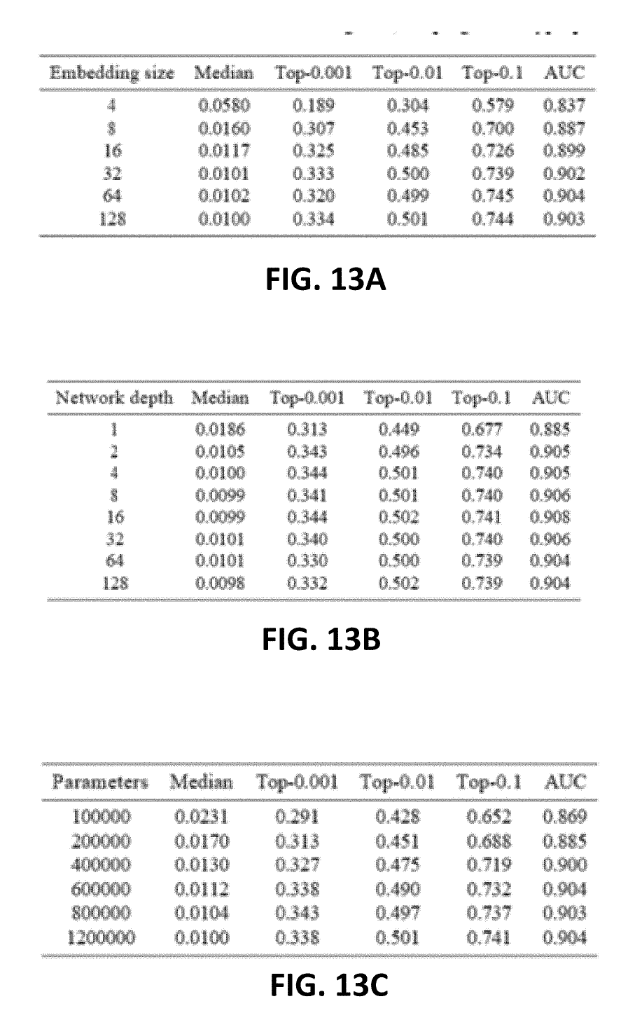

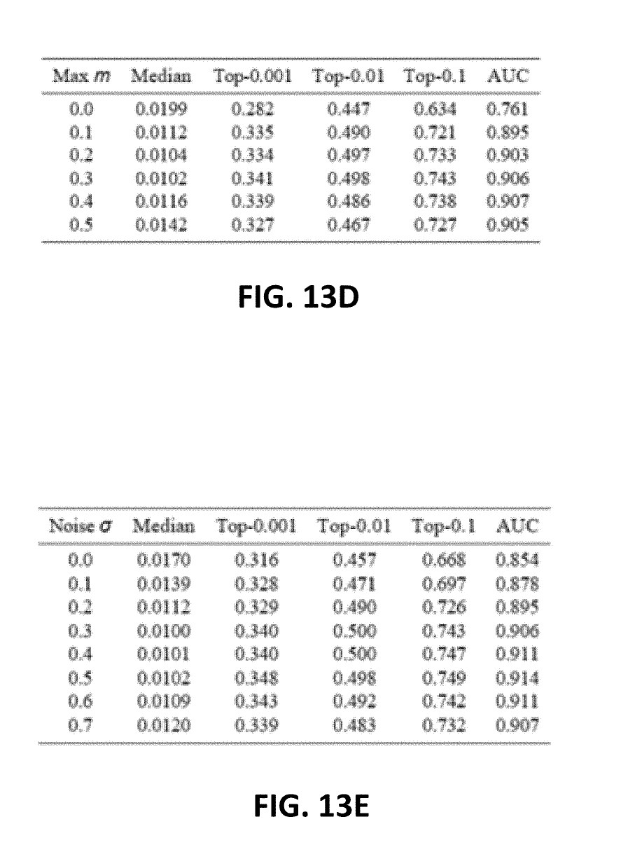

FIG. 13A is a table of performance as a function of embedding size, keeping other hyperparameters fixed.

FIG. 13B is a table of performance as a function of the network depth, keeping other hyperparameters fixed.

FIG. 13C is a table of performance as a function of the number of trainable parameter changes, keeping other hyperparameter fixed.

FIG. 13D is a table of performance as a function of the maximum value of the margin parameter, keeping other hyperparameters fixed.

FIG. 13E is a table of performance as a function of standard deviation of the added noise changes, keeping other hyperparameters fixed.

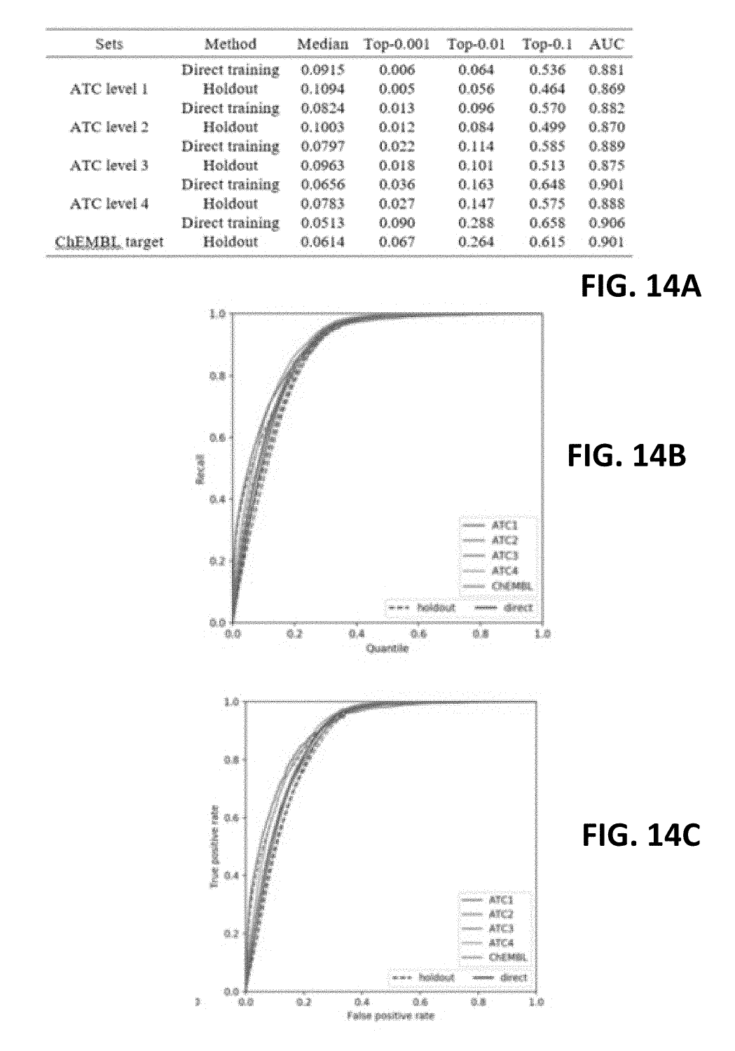

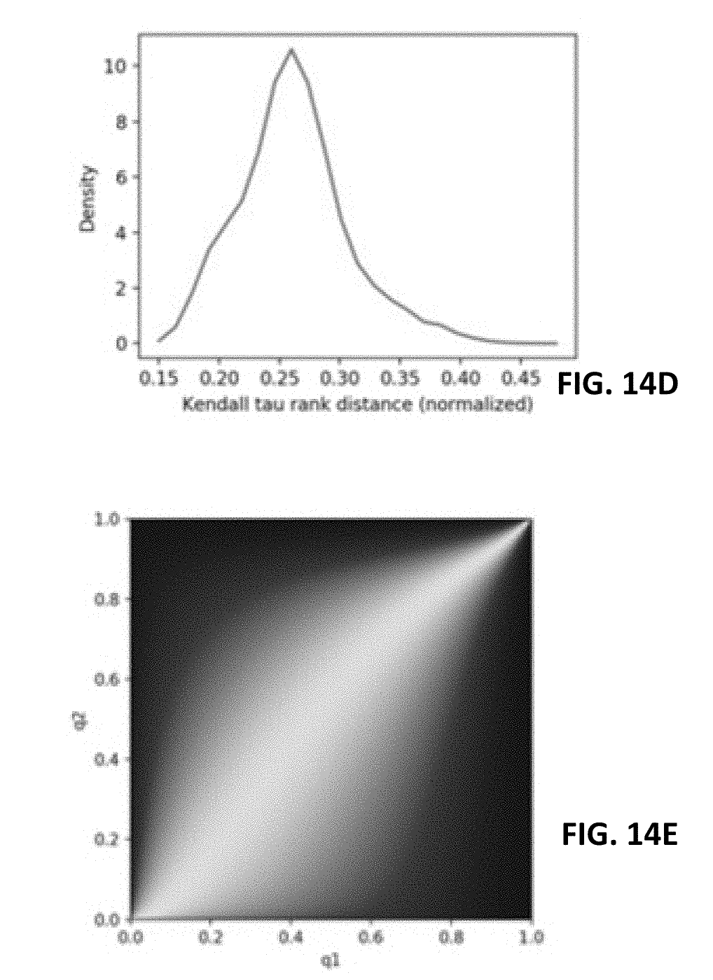

FIG. 14A is a table of a comparison of two methods of computing perturbagen embeddings: direct training and holdout. The comparison was performed on ATC levels 1-4 and ChEMBL protein targets.

FIG. 14B is a graph of quantile vs. recall for the direct training and holdout methods of computing embeddings.

FIG. 14C is a graph of ROC plots of the direct training and hold-out methods of computing embeddings.

FIG. 14D is a graph of estimated density of normalized Kendall distances between direct training and holdout similarities

FIG. 14E is a heatmap histogram of quantiles of similarity using the holdout method q₂ for each quantile of similarity using the direct training method q₁.

### VII. Additional Considerations

It is to be understood that the figures and descriptions of the present disclosure have been simplified to illustrate elements that are relevant for a clear understanding of the present disclosure, while eliminating, for the purpose of clarity, many other elements found in a typical system. Those of ordinary skill in the art may recognize that other elements and/or steps are desirable and/or required in implementing the present disclosure. However, because such elements and steps are well known in the art, and because they do not facilitate a better understanding of the present disclosure, a discussion of such elements and steps is not provided herein. The disclosure herein is directed to all such variations and modifications to such elements and methods known to those skilled in the art.

Some portions of above description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

While particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims

What is claimed is:

1. A method comprising:
receiving, for a query perturbagen, an expression profile including transcription counts of a plurality of genes in a cell affected by the query perturbagen;
inputting the expression profile for the query perturbagen into a trained model to extract an embedding comprising an array of features comprising corresponding feature values,
determining, based on the extracted embedding, a similarity score between the query perturbagen and each of a plurality of known perturbagens, the similarity score describing a likelihood that a known perturbagen has a similar effect on gene expression in a cell as the query perturbagen;
ranking each of the similarity scores based on the likelihoods; and
determining, from the ranked similarity scores, at least one candidate perturbagen that matches the query perturbagen.

2. The method of claim 1, wherein the model is a deep neural network.

3. The method of claim 1, wherein the model comprises a plurality of layers, wherein the layers are densely connected without a convolution.

4. The method of claim 3, wherein the model comprises a final fully-connected layer which computes an un-normalized embedding.

5. The method of claim 3, wherein the model further comprises a layer following the final fully-connected layer which computes a normalized embedding.

6. The method of claim 1, wherein the model is trained using a modified softmax cross-entropy loss over n identities of known perturbagens.

7. The method of claim 6, wherein determining the loss over each known perturbagen comprises:
computing a cosine of an angle between an embedding vector and a class weight, wherein the cosine of the angle is computed according to:
cos θᵢ=(Wᵢ^{T}x)/∥Wᵢ∥₂∥x∥₂ to.

8. The method of claim 6, wherein determining the loss over each known perturbagen comprises:
computing the loss over each known perturbagen as a function of the computed cosine of the angle.

9. The method of claim 8, wherein the loss is computed according to:
$$l ( θ ) = - log \frac{exp ( ∝ ( cos {θ}_{i} - m ) )}{exp ( ∝ ( cos {θ}_{i} - m ) ) + {\sum }_{j \neq i} exp ( ∝ cos {θ}_{j} )} .$$

10. The method of claim 1, wherein the expression profile for the query perturbagen comprises a transcript count for each gene measured using L1000 Expression Profiling.

11. The method of claim 1, wherein known perturbagens are labeled with an identity of a compound.

12. The method of claim 1, wherein determining the similarity score between the query perturbagen and a known perturbagen comprises:
computing a cosine similarity between each embedding of the set of known perturbagens and the embedding of the query perturbagen.

13. The method of claim 1, wherein determining the similarity score between the query perturbagen and a known perturbagen comprises:
computing a Euclidean distance between each embedding of the set of known perturbagens and the embedding of the query perturbagen.

14. The method of claim 1, wherein the model is trained using a training dataset of expression profiles for known perturbagens, each known perturbagen associated with a pharmacological effect on gene expression within a cell.

15. The method of claim 14, wherein a known perturbagen is further associated with a therapeutic target classification.

16. The method of claim 14, wherein a known perturbagen is further associated with a molecular structure of a compound.

17. The method of claim 1, further comprising:
determining, for a query perturbagen, an aggregate embedding, wherein the aggregate expression profile is an average of embeddings for a plurality of expression profiles associated with the query perturbagen;
determining, based on the aggregate embedding, a similarity score between the query perturbagen and each of plurality of known perturbagens, wherein each known perturbagen is associated with an aggregate embedding and each similarity score indicates a likelihood that a known perturbagen has a similar effect on gene expression in a cell as the query perturbagen;
ranking each similarity score; and
determining, from the ranked similarity scores, at least one candidate perturbagen that matches the query perturbagen.

18. The method of claim 1, wherein the model is trained using a training dataset of expression profiles for a plurality of known perturbagens, the training dataset comprising:
for each known perturbagen, a first subset of expression profiles associated with the known perturbagen; and
a second subset of the remaining expression profiles associated with a different one of the known perturbagens.

19. The method of claim 18, wherein the first subset of expression profiles associated with the known perturbagen comprises expression profiles for biological replicates of the known perturbagen.

20. The method of claim 19, wherein the biological replicates and the known perturbagen share a cell line, a dosage, and a time at which the dosage was applied.

21. The method of claim 1, wherein determining at least one candidate perturbagen comprises:
selecting, based on their ranked similarity score, a candidate perturbagen.

22. The method of claim 1, wherein determining at least one candidate perturbagen comprises:
accessing, from computer memory, a threshold rank; and
selecting at least one of the ranked similarity scores above the threshold rank, wherein each selected similarity score represents a candidate perturbagen.

23. The method of claim 1, wherein determining at least one candidate perturbagen comprises:
accessing, from computer memory, a threshold similarity score; an
selecting at least one similarity score of the embedding above the threshold similarity score, wherein each selected similarity score represents a candidate perturbagen.

24. The method of claim 1, wherein the model is verified using a subset of expression profiles from the training dataset expressions excluded from the training of the model, the verification comprising:
for a known perturbagen, inputting an expression profile of the excluded subset to the trained model to extract an embedding;
determining a level of similarity between the extracted embedding and embeddings for a first subset of expression profiles of the same perturbagen;
determining a level of similarity between the extracted embedding and embeddings for a second subset of expression profiles of different perturbagens;
confirming, from the first and second subsets, that the embedding with the highest level of similarity to the extracted embedding belongs to the first subset of expression profiles.

25. The method of claim 24, wherein the first subset of expression profiles comprises biological replicates of the same perturbagen and the second subset of expression profiles comprise expression profiles of different perturbagens and expression profiles of non-biological replicates of the same perturbagen.

26. The method of claim 1, further comprising:
determining a set of functional properties associated with each of the candidate perturbagens, wherein functional properties describe a pharmacological effect on gene expression in a cell; and
assigning one or more of the functional properties to the query perturbagen.

27. The method of claim 1, further comprising:
accessing, from a database of known perturbagens, at least one candidate perturbagen that matches the query perturbagen, wherein the candidate perturbagen is associated with at least one functional property;
assigning, based on the candidate perturbagens, at least one functional property to the query perturbagen; and
storing, within the database of known perturbagens, the query perturbagen with the assigned functional properties.

28. The method of claim 1, wherein shared functional properties are categorized based on levels of anatomical therapeutic target classifications.

29. A method comprising:
accessing, from a database of known perturbagens, a set of known perturbagens, wherein each known perturbagens is associated with at least one functional property describing an effect on gene expression in a cell;
selecting, from the set of known perturbagens, a first perturbagen as a query perturbagen;
accessing, for the query perturbagen, an embedding comprising an array of features comprising corresponding feature values;
determining, based on the extracted embedding, a similarity score between the query perturbagen and each of a plurality of known perturbagens, the similarity score describing a likelihood that a known perturbagen of the accessed set has a similar effect on gene expression in a cell as the query perturbagen;
determining, from the similarity scores, at least one candidate perturbagen that matches the query perturbagen; and
supplementing a set of functional properties associated with query perturbagen with one or more functional properties associated with the candidate perturbagens.

30. The method of claim 29, further comprising:
accessing, for the query perturbagens, an aggregate embedding based on an average of embeddings for a plurality of expression profiles associated with the query perturbagen; and
determining, from the aggregate embedding, at least one candidate perturbagen that matches the query perturbagen.

31. The method of claim 29, wherein supplementing the functional properties associated with the query perturbagen comprises:
determining a measure of structural similarity based on the functional properties associated with the query perturbagen; and
supplementing a set of structural properties associated with the query perturbagen with one or more structural properties associated with the candidate perturbagen.

32. The method of claim 31, wherein the measure of structural similarity is determined based on a Tanimoto coefficient, the determination comprising:
computing, for each candidate perturbagen, a Tanimoto coefficient representing the structural similarity between the query perturbagen and the candidate perturbagen;
responsive to determining that the Tanimoto coefficient is greater than a threshold value, determining that the query perturbagen is structurally similar to the candidate perturbagen; and
storing the Tanimoto coefficient in computer memory.

33. The method of claim 32, wherein the Tanimoto coefficient is computed based on one or more of the following:
an extended-connectivity fingerprint key; and
a Molecular ACCess System key.

34. The method of claim 31, wherein the measure of structural similarity is determined based on the extracted embedding of the query perturbagen and a Tanimoto coefficient of a candidate perturbagen, the determination comprising:
accessing the extracted embedding for the query perturbagen and the Tanimoto coefficient for the candidate perturbagen from computer memory;
determining the similarity score corresponding to the likelihood of functional similarity between the query perturbagen and the candidate perturbagen from the extracted embedding;
determining an average of the similarity score of the candidate perturbagen and the Tanimoto coefficient of the candidate perturbagen; and
responsive to determining that the average is greater than a threshold value, determining at least one structural property associated with the query perturbagen.

35. The method of claim 31, wherein the measure of structural similarity is determined based on the extracted embedding of the query perturbagen and a Tanimoto coefficient of a candidate perturbagen, the determination comprising:
accessing the extracted embedding for the query perturbagen and the Tanimoto coefficient for the candidate perturbagen from computer memory;
determining the similarity score corresponding to the likelihood of functional similarity between the query perturbagen and the candidate perturbagen from the extracted embedding;
determining an combination of the similarity score of the candidate perturbagen and the Tanimoto coefficient of the candidate perturbagen; and
responsive to determining that the average is greater than a threshold value, determining at least one structural property associated with the query perturbagen.

36. The method of claim 29, further comprising:
increasing the rank of candidate perturbagens which are structurally similar to the query perturbagen; and
reducing the rank of candidate perturbagens which are not structurally similar to the query perturbagen.

37. The method of claim 29, further comprising:
performing, for a query perturbagen, a wet-lab simulation, wherein the simulation applies the query perturbagen to a plurality of cells;
measuring, based on the wet-lab simulation, transcription counts of a plurality of genes in each cell of the plurality;
determining, from the transcription counts of each cell, one or more biological targets within the cell affected by the query perturbagen; and
generating, based on the transcription counts, an expression profile for the query perturbagen.

38. The method of claim 37, further comprising:
measuring, for each of a plurality of known perturbagens, transcription counts of a plurality of genes in cells affected by the known perturbagen;
determining, from the transcription counts of each cell, one or more biological targets within the cell affected by the known perturbagen;
comparing the biological targets affected by the query perturbagen to the biological targets affected by the known perturbagen; and
for biological targets shared between the query perturbagen and the known perturbagen, relating the query perturbagen to the known perturbagen.

39. A system comprising:
a processor; and
a non-transitory computer readable storage medium comprising computer program instructions that when executed by a computer processor cause the processor to:
receive, for a query perturbagen, an expression profile including transcription counts of a plurality of genes in a cell affected by the query perturbagen;
input the expression profile for the query perturbagen into a trained model to extract an embedding comprising an array of features comprising corresponding feature values,
determine, based on the extracted embedding, a similarity score between the query perturbagen and each of a plurality of known perturbagens, the similarity score describing a likelihood that a known perturbagen has a similar effect on gene expression in a cell as the query perturbagen;
rank each of the similarity scores based on the likelihoods; and
determine, from the ranked similarity scores, at least one candidate perturbagen that matches the query perturbagen.
